(19) United States
(12) Patent Application Publication
(10) Pub. No.: US 2023/0043806 A1
(43) Pub. Date: Feb. 9, 2023

(54) INDUCTION OF HIGHLY EFFICACIOUS ANTI-TUMOR AND IMMUNE MODULATING ACTIVITY: CELL-FREE OFF THE SHELF THERAPEUTIC MODALITY

(71) Applicants: University of Virginia Patent Foundation, Charlottesville, VA (US); Wayne State University, Detroit, MI (US)

(72) Inventors: Archana Thakur, Zion Crosseroads (US); Lawrence G. Lum, Charlottesville (US); Srividya Yarlagadda, Rochester (US)

(21) Appl. No.: 17/434,692

(22) PCT Filed: Feb. 28, 2020
(86) PCT No.: PCT/US2020/020476

§ 371 Date: Aug. 27, 2021

Related U.S. Application Data
(60) Provisional application No. 62/811,639, filed on Feb. 28, 2019.

(51) Int. Cl. A61K 35/17; A61K 35/13; C07K 16/28; A61P 35/00

(52) U.S. Cl. CPC A61K 35/17; A61K 35/13; C07K 16/2809; A61P 35/00

(57) ABSTRACT

Methods and compositions for treating cancer in a subject in need thereof. The method includes administering to the subject an effective amount of a composition comprising Tumor-Targeting Effectors (TITE) derived from a culture comprising a bispecific antibody armed activated T cell (BAT) and a cancer cell, to thereby treat cancer in the subject.

### CROSS REFERENCE TO RELATED APPLICATION

The presently disclosed subject matter claims the benefit of U.S. Provisional Patent Application Ser. No. 62/811,639, filed Feb. 28, 2019; the disclosure of which is incorporated herein by reference in its entirety.

### GOVERNMENT INTEREST

This invention was made with government support under Grant Nos. CA092344, CA140314, and CA182526, awarded by The National Institutes of Health. The government has certain rights in the invention.

### BACKGROUND

Most therapeutic approaches for cancer are based on targeting a tumor or a single component of the tumor-supporting microenvironment that eventually results in cancer recurrence. Thus, there is a long felt need in the art for compositions and methods useful for treating cancer. The presently disclosed subject matter addresses this need and other needs in the art.

### SUMMARY

This Summary lists several embodiments of the presently disclosed subject matter, and in many cases lists variations and permutations of these embodiments of the presently disclosed subject matter. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

In some embodiments, the presently disclosed subject matter provides a method for treating cancer in a subject in need thereof. In some embodiments, the method comprises administering to the subject an effective amount of a composition comprising Tumor-Targeting Effectors (TITE) derived from a culture comprising a bispecific antibody (BiAb) armed activated T cell (BAT) and a cancer cell, to thereby treat cancer in the subject. In some embodiments, the composition comprising TITE is derived from an about 16 hour to an about 48 hour culture comprising a bispecific antibody armed activated T cell (BAT) and a cancer cell. In some embodiments, the cancer cell is from a cancer selected from the group consisting of a breast cancer, a pancreatic cancer, a prostate cancer, a brain cancer, a lung cancer, an ovarian cancer, a liver cancer, a leukemia, non-Hodgkin's lymphoma and multiple myeloma. In some embodiments, the method further comprises administering an additional therapeutic agent.

In some embodiments, the composition comprising TITE is derived from a culture comprising BiAb armed activated T cells and tumor cells at a T cell effector:tumor cell ratio ranging from about 10:1 to about 50:1. In some embodiments, the composition comprising TITE is derived from a culture comprising BiAb armed activated T cells and tumor cells in a medium supplemented with a serum at a range of about 2% to about 10% by weight or by volume. In some embodiments, the BiAb used to arm the activated T cell is a chemically heteroconjugated bispecific antibody or a recombinant bispecific antibody of any configuration. In some embodiments, the activated T cells are produced from an apheresis product. In some embodiments, the activated T cells are produced from an apheresis product by anti-CD3 stimulation in the presence of IL-2, optionally at a range of about 20 to about 200 IU/ml, or wherein co-stimulated T cells are produced from an apheresis product by co-stimulation with anti-CD³/anti-CD28 coated beads, optionally in the presence of IL-2 at a range of about 20 to about 200 IU/ml, optionally at bead to cell ratios from about 1:3 to about 3:1.

In some embodiments, the culture comprises peripheral blood mononuclear cells, unfractionated CD3+ T cells, CD4+ T cells, or CD8+ T cells. In some embodiments, the TITE comprise secreted agents selected from the group consisting of a Th1 cytokine, a proliferation inducing cytokine, a Th2 cytokine, a chemokine, a growth factor, and any combination thereof. In some embodiments, the Th1 cytokine is selected from the group consisting of IFN-γ, TNF-α, Granzyme B, GM-CSF, G-CSF, and combinations thereof; the proliferation inducing cytokine is selected from the group consisting of Flt3L, IL-2, IL-3, and combinations thereof the Th2 cytokine is selected from the group consisting of IL-10, IL-5, IL-6, IL-13, and combinations thereof; and/or the growth factor is selected from the group consisting of CD40L, VEGF, PDGF-AA, and combinations thereof.

In some embodiments, the composition comprising TITE comprises an amount of TITE ranging from about 5% to about 50% by weight or by volume. In some embodiments, the composition comprising TITE comprises a fraction comprising components of a molecular weight ranging from about 10 kiloDaltons (kDa) to about 50 kDa.

In some embodiments, the composition comprising TITE comprises a miRNA. In some embodiments, the miRNA is selected from the group consisting of miR-16-5p, miR-17-5p, miR-195-5p, miR-20a-5p, miR-93-5p, miR-155, miR-181a-5p, miR-181c-5p, miR186-5p, and miR-106a-5p.

In some embodiments, the presently disclosed subject matter provides the use of a pharmaceutical composition comprising, consisting essentially of, or consisting of an effective amount of a composition comprising Tumor-Targeting Effectors (TITE) derived from a culture comprising a bispecific antibody armed activated T cell (BAT) and a cancer cell to treat cancer in a subject in need thereof.

In some embodiments, the presently disclosed subject matter provides the use of an effective amount of a composition comprising Tumor-Targeting Effectors (TITE) derived from a culture comprising a bispecific antibody armed activated T cell (BAT) and a cancer cell for the preparation of a medicament to treat cancer in a subject in need thereof.

In some embodiments, the presently disclosed subject matter provides a pharmaceutical composition comprising, consisting essentially of, or consisting of an effective amount of a composition comprising Tumor-Targeting Effectors (TITE) derived from a culture comprising a bispecific antibody armed activated T cell (BAT) and a cancer cell.

In some embodiments, the composition comprising TITE is derived from an about 16 hour to an about 48 hour culture comprising a bispecific antibody armed activated T cell (BAT) and a cancer cell. In some embodiments, the cancer cell is from a cancer selected from the group consisting of a breast cancer, a pancreatic cancer, a prostate cancer, a brain cancer, a lung cancer, an ovarian cancer, a liver cancer, a leukemia, non-Hodgkin's lymphoma and multiple myeloma. In some embodiments, the method further comprises administering an additional therapeutic agent.

In some embodiments, the composition comprising TITE is derived from a culture comprising BiAb armed activated T cells and tumor cells at a T cell effector:tumor cell ratio ranging from about 10:1 to about 50:1. In some embodiments, the composition comprising TITE is derived from a culture comprising BiAb armed activated T cells and tumor cells in a medium supplemented with a serum at a range of about 2% to about 10% by weight or by volume. In some embodiments, the BiAb used to arm the activated T cell is a chemically heteroconjugated bispecific antibody or a recombinant bispecific antibody of any configuration. In some embodiments, the activated T cells are produced from an apheresis product. In some embodiments, the activated T cells are produced from an apheresis product by anti-CD3 stimulation in the presence of IL-2, optionally at a range of about 20 to about 200 IU/ml, or wherein co-stimulated T cells are produced from an apheresis product by co-stimulation with anti-CD³/anti-CD28 coated beads, optionally in the presence of IL-2 at a range of about 20 to about 200 IU/ml, optionally at bead to cell ratios from about 1:3 to about 3:1.

In some embodiments, the culture comprises peripheral blood mononuclear cells, unfractionated CD3+ T cells, CD4+ T cells, or CD8+ T cells. In some embodiments, the TITE comprise secreted agents selected from the group consisting of a Th1 cytokine, a proliferation inducing cytokine, a Th2 cytokine, a chemokine, a growth factor, and any combination thereof. In some embodiments, the Th1 cytokine is selected from the group consisting of IFN-γ, TNF-α, Granzyme B, GM-CSF, G-CSF, and combinations thereof; the proliferation inducing cytokine is selected from the group consisting of Flt3L, IL-2, IL-3, and combinations thereof; the Th2 cytokine is selected from the group consisting of IL-10, IL-5, IL-6, IL-13, and combinations thereof; and/or the growth factor is selected from the group consisting of CD40L, VEGF, PDGF-AA, and combinations thereof.

In some embodiments, the composition comprising TITE comprises an amount of TITE ranging from about 5% to about 50% by weight or by volume. In some embodiments, the composition comprising TITE comprises a fraction comprising components of a molecular weight ranging from about 10 kiloDaltons (kDa) to about 50 kDa.

In some embodiments, the composition comprising TITE comprises a miRNA. In some embodiments, the miRNA is selected from the group consisting of miR-16-5p, miR-17-5p, miR-195-5p, miR-20a-5p, miR-93-5p, miR-155, miR-181a-5p, miR-181c-5p, miR186-5p, and miR-106a-5p.

In some embodiments, the use or composition further comprises an additional therapeutic agent.

In some embodiments, the presently disclosed subject matter provides a method of preparing a composition for treating cancer, the method comprising (a) culturing T cells and cancer cells with a bispecific antibody to provide a culture comprising a complex comprising cancer cells and activated T cells (ATC) wherein one binding domain of the bispecific antibody binds to an antigen on the T cells and a second binding domain of the bispecific antibody binds an antigen on the cancer cells; and (b) isolating media from the culture, wherein the media comprises BAT Induced Tumor-Targeting Effectors (TITE), to thereby provide a composition for treating cancer. A composition for treating cancer produced by the method is also provided.

In some embodiments, the composition comprising TITE is derived from an about 16 hour to an about 48 hour culture comprising a bispecific antibody armed activated T cell (BAT) and a cancer cell. In some embodiments, the cancer cell is from a cancer selected from the group consisting of a breast cancer, a pancreatic cancer, a prostate cancer, a brain cancer, a lung cancer, an ovarian cancer, a liver cancer, a leukemia, non-Hodgkin's lymphoma and multiple myeloma. In some embodiments, the method further comprises administering an additional therapeutic agent.

In some embodiments, the composition comprising TITE is derived from a culture comprising BiAb armed activated T cells and tumor cells at a T cell effector:tumor cell ratio ranging from about 10:1 to about 50:1. In some embodiments, the composition comprising TITE is derived from a culture comprising BiAb armed activated T cells and tumor cells in a medium supplemented with a serum at a range of about 2% to about 10% by weight or by volume. In some embodiments, the BiAb used to arm the activated T cell is a chemically heteroconjugated bispecific antibody or a recombinant bispecific antibody of any configuration. In some embodiments, the activated T cells are produced from an apheresis product. In some embodiments, the activated T cells are produced from an apheresis product by anti-CD3 stimulation in the presence of IL-2, optionally at a range of about 20 to about 200 IU/ml, or wherein co-stimulated T cells are produced from an apheresis product by co-stimulation with anti-CD³/anti-CD28 coated beads, optionally in the presence of IL-2 at a range of about 20 to about 200 IU/ml, optionally at bead to cell ratios from about 1:3 to about 3:1.

In some embodiments, the culture comprises peripheral blood mononuclear cells, unfractionated CD3+ T cells, CD4+ T cells, or CD8+ T cells. In some embodiments, the TITE comprise secreted agents selected from the group consisting of a Th1 cytokine, a proliferation inducing cytokine, a Th2 cytokine, a chemokine, a growth factor, and any combination thereof. In some embodiments, the Th1 cytokine is selected from the group consisting of IFN-γ, TNF-α, Granzyme B, GM-CSF, G-CSF, and combinations thereof; the proliferation inducing cytokine is selected from the group consisting of Flt3L, IL-2, IL-3, and combinations thereof the Th2 cytokine is selected from the group consisting of IL-10, IL-5, IL-6, IL-13, and combinations thereof; and/or the growth factor is selected from the group consisting of CD40L, VEGF, PDGF-AA, and combinations thereof.

In some embodiments, the composition comprising TITE comprises an amount of TITE ranging from about 5% to about 50% by weight or by volume. In some embodiments, the composition comprising TITE comprises a fraction comprising components of a molecular weight ranging from about 10 kiloDaltons (kDa) to about 50 kDa.

In some embodiments, the composition comprising TITE comprises a miRNA. In some embodiments, the miRNA is selected from the group consisting of miR-16-5p, miR-17-5p, miR-195-5p, miR-20a-5p, miR-93-5p, miR-155, miR-181a-5p, miR-181c-5p, miR186-5p, and miR-106a-5p.

In some embodiments of the method, use, or composition of the presently disclosed subject matter, the composition comprising TITE is adapted for administration for the treatment of a subject by intravenous administration, intrathecal injection, peritoneal injection, or direct injection into the tumor or surround tumor site. In some embodiments, the subject is a mammalian subject.

Accordingly, it is an object of the presently disclosed subject matter to provide compositions and methods for treating cancer. This and other objects are achieved in whole or in part by the presently disclosed subject matter. Further, objects of the presently disclosed subject matter having been stated above, other objects and advantages of the presently disclosed subject matter will become apparent to those skilled in the art after a study of the following description, Figures, and EXAMPLES. Additionally, various aspects and embodiments of the presently disclosed subject matter are described in further detail below.

### BRIEF DESCRIPTION OF THE FIGURES

FIGS. 1A-1E are a series of images showing the effect of TITE on breast cancer-BT-20 tumor spheres assessed in 3D culture. Cytotoxic activity was assessed under microscopy at dose levels ranging from 0%, 10%, 25%, 50%, 75%, and 100% of TITE against tumor spheres (BT20) presented. TITE was prepared from T cell subpopulations, CD3+ BATs+tumor(T) (FIG. 1A); CD4+ BATs+T (FIG. 1B), and CD8+ BATs+T (FIG. 1C). Cytotoxicity at BT20 tumor spheres+BISE co-cultured with MDSC was also observed as compared to a BT20 only control and BT20+MDSC (FIG. 1D). Cytotoxic activity of MiaPaCa-2 tumor spheres co-cultured with BATs or PBMC in the presence or absence of TITE was also assessed, using cultures comprising Mia alone, Mia+BAT, Mia+PMBC, Mia+10% BISE, Mia+BAT (FIG. 1E). FIG. 1F is a bar graph showing total volume of LECs. FIG. 1G is a bar graph showing that the soluble factor(s) between 10 to 50 kDa molecular weight retain cytotoxic activity. FIG. 1H shows that in the presence of TITE, proportion of CD133+/EpCAM/+CD44hi/CD2410 CSC was reduced to 0.7% compared to 4.9% in control culture without TITE or cultures containing B-CM (2.1%). FIG. 1I is a series of graphs showing the effect of TITE on solid tumor cell lines at 5, 10 and 25% concentration against solid tumor cell lines. These findings were confirmed in a larger number of cancer cell lines. At 25% concentration of TITE, highly significant cytotoxicity (p<0.05-p<0.0005) was observed against MB231, MCF-7, SKBR3, MiaPaCa-2, L3.6p1, CoLo-356, HCT8, H292, A549, HN6 compared to B-CM at 72 hours compared to B-CM. FIG. 1J is a series of graphs showing the cytotoxicity by TITE and BATs against breast and pancreatic cancer cell lines (top graphs) using real time cell analysis (RTCA). Lower graphs show enhanced cytotoxicity by BATs against TITE primed MB231 and MiaPaCa-2 cells over 120 hours.

FIGS. 2A-2E show immune cell modulation by TITE or B-CM. FIG. 2A is a set of bar graphs showing that normal donor PBMC incubated with control B-CM or TITE show CD4+/CD69+ and CD8+/CD69+ activated T cells in the absence of tumor cells (FIG. 2A, left panel). FIG. 2A, right panel shows activated CD4+/CD69+ and CD8+/CD69+ T cells, CD33+/HLA−DR−, CD4+/CD25+/CD12710 Treg cells in the presence of MB231 tumor cells. In the left panel of FIG. 2A, the left hand bar of each touching pair of bars corresponds to data for CD4+/CD69+ activated T cells, while each right hand bar of each touching pair of bars corresponds to data for CD8+/CD69+ activated T cells. In the right panel of FIG. 2A, the bars for each set of four touching bars correspond, from left to right, to data for CD4+/CD69+ T cells, CD8+/CD69+ T cells, CD33+/HLA−DR− cells, and CD4+/CD25+/CD12710 Treg cells. FIG. 2B is a series of bar graphs showing the expression of co-stimulatory (4-1BB, ICOS and OX40) or co-inhibitory (PD-1) markers on CD4 (FIG. 2B, upper panel) and CD8 T (FIG. 2B, lower panel) cells in the co-cultures of MB231 or MCF-7 and PBMC for 48 h with various percentages of TITE. For the upper panel of FIG. 2B, data is grouped in sets of four bars for each culture condition corresponding, from left to right, to CD4+/41BB+. CD4+/ICOS+, CD4+/OX40+, and CD4+/PD1+ cells. For the lower panel of FIG. 2b, data is grouped in sets of four bars for each culture condition corresponding, from left to right, to CD8+/41BB+, CD8+/ICOS+, CD8+/OX40+, and CD8+/PD1+ cells. FIG. 2C is a heat map showing the quantitative cytokine profiles of BISE and control BISE (T-CM and B-CM) using 45-panel using Luminex multiplex technology. The heat map shows the representative profiles of all three BISEs. FIG. 2C, left panel shows the profile of TITE prepared from MB231+ BATs and FIG. 2C, right panel shows the heat map of TITE prepared from MiaPaCa-2+ BATs. The panels show different levels of particular TITE, but the profile remains essentially the same. FIGS. 2D-1 through 2D-6 show the quantitative distribution of cytokines (FIGS. 2D-1 through 2D-3) and chemokines/growth factors (FIGS. 2D-4 through 2D-6) in pg/ml. FIG. 2E is a series of graphs showing the effect of key cytokines on cytotoxicity of MB231 cells. IFN-γ and TNF-α both induced cytotoxicity. However, IFN-γ (left hand bar in each set of three touching bars) showed significantly increased cytotoxicity compared to TNF-α (middle bar in each set of three touching bars) and the combination of both IFN-γ and TNF-α (right hand bar in each set of three touching bars) showed an additive effect on cytolytic activity (FIG. 2E, bottom panel). IL-6 and GM-CSF both showed no cytotoxic effects on tumor cells.

FIG. 3A is a heat map showing the signaling array of TITE and controls (T-CM and B-CM). The heat map shows the relative fold change profiles of all three CMs compared to internal control GAPDH. FIG. 3B is a blot showing the validation of selected signaling proteins by western blot. FIG. 3C shows the miRNA array of TITE and control CMs (T-CM and B-CM). FIG. 3D and FIG. 3E are a heat map and a graph, respectively, showing an average fold change in miRNA isolated from exosomes in B-CM and TITE relative to T-CM prepared from 3 normal donor BATs. FIG. 3F is a series of bar graphs showing the validation of miR93, miR-155, mi-R21, miRlet-7, miR-34a, miR-15a, miR-150 and miR-145a by qRT-PCR.

FIGS. 4A-4C are a series of graphs showing the evaluation of the injection of the MB-231 breast cancer cell line into the flanks of ICR-SCID mice (n=5/group). Tumor-bearing mice were treated with IV injections of BATs, TITE and vehicle 2×/week for 3 weeks (FIG. 4A). FIG. 4B shows that tumor-bearing mice treated with IV vs IT injections of TITE or vehicle 3×/week for 3 weeks show significant delay in tumor volume (p<0.003), while FIG. 4C shows that IV treatment resulted in significant reduction in as tumor size (p<0.05), as well.

FIG. 5A is a schematic figure showing the proposed mechanism of intracellular signaling triggered by the engagement of BATs with tumor cells via bispecific antibodies. FIG. 5B is a schematic figure showing the proposed mechanism of action of TITE in the TME immune modulation and generation of in situ immunization at the cellular level through cytokines/chemokines and microRNA.

### DETAILED DESCRIPTION

Headings are included herein for reference and to aid in locating certain sections. These headings are not intended to limit the scope of the concepts described therein under, and these concepts can have applicability in other sections throughout the entire specification.

Adoptive transfer of Bispecific antibody Armed activated T cells (BATs) show promising anti-tumor activity in clinical trials in solid tumors. The cytotoxic activity of BATs occurs upon engagement with tumor cells via the bispecific antibody bridge which stimulates BATs to release not only lytic and cytotoxic molecules (perforin/granzyme) but also cytokines, chemokines, and other signaling molecules extracellularly. It was hypothesized that the release of BAT induced Tumor-Targeting Effectors (TITE) by this complex interaction of T cells, bispecific antibody, and tumor cells can serve as a potent anti-tumor and immune activating immunotherapeutic approach. In a 3D tumor sphere model, TITE showed a potent cytotoxic activity against multiple breast (MDA-MB-231, BT-20, SKBR-3 and MCF-7) cancer cell lines compared to control conditioned media (CM), Tumor-CM (T-CM) or BAT-CM (B-CM). Multiplex analysis showed high levels of Th1 cytokine and chemokines, and a phospho-protein signaling array showed prominent JAK1/STAT1/STAT5A that can be responsible for the induction and release of Th1 cytokines/chemokines in TITE. Exosomal microRNA (miR) in TITE showed higher expression of several miRs that are associated with T cell function and activation compared to control CMs. In a xenograft breast cancer model, IV injections of 10× concentrated TITE (3×/week for 3 weeks; 150p1 TITE/injection) was able to inhibit tumor growth significantly (p<0.003) compared to the control mice. In summary, BATs-Tumor complex derived TITE provides a clinically controllable cell-free platform to target various tumor types with diverse anti-cancer immune activating mediators regardless of the heterogeneous nature of the tumor cells and mutational burden as a novel and potent off-the-shelf therapeutic modality. Therapeutic advantages of TITE include but are not limited to: 1) a ready off-the-shelf product; 2) a decrease in regulatory and manufacturing costs.

### I. Abbreviations and Acronyms

ATC—activated T cells
BAT—Bispecific antibody Armed activated T cell
BiAb—bispecific antibodies
CM—control conditioned media
MDSC—myeloid-derived suppressor cell(s)
PBMC—peripheral blood mononuclear cell(s)

### TITE—Tumor-Targeting Effector(s)

### TME—Tumor Microenvironment

### II. Definitions

In describing and claiming the presently disclosed subject matter, the following terminology will be used in accordance with the definitions set forth below.

The articles “a” and “an” are used herein to refer to one or to more than one (i.e., to at least one) of the grammatical object of the article. By way of example, “an element” means one element or more than one element.

The term “about”, as used herein, means approximately, in the region of, roughly, or around. When the term “about” is used in conjunction with a numerical range, it modifies that range by extending the boundaries above and below the numerical values set forth. For example, in some embodiments, the term “about” is used herein to modify a numerical value above and below the stated value by a variance of 10%. Therefore, about 50% means in the range of 45%-55%. Numerical ranges recited herein by endpoints include all numbers and fractions subsumed within that range (e.g., 1 to 5 includes 1, 1.5, 2, 2.75, 3, 3.90, 4, and 5). It is also to be understood that all numbers and fractions thereof are presumed to be modified by the term “about”.

As used herein, the phrase “biological sample” refers to a sample isolated from a subject (e.g., a biopsy, blood, serum, etc.) or from a cell or tissue from a subject (e.g., RNA and/or DNA and/or a protein or polypeptide isolated therefrom). Biological samples can be of any biological tissue or fluid or cells from any organism as well as cells cultured in vitro, such as cell lines and tissue culture cells. Frequently the sample will be a “clinical sample” which is a sample derived from a subject (i.e., a subject undergoing a diagnostic procedure and/or a treatment). Typical clinical samples include, but are not limited to cerebrospinal fluid, serum, plasma, blood, saliva, skin, muscle, olfactory tissue, lacrimal fluid, synovial fluid, nail tissue, hair, feces, urine, a tissue or cell type, and combinations thereof, tissue or fine needle biopsy samples, and cells therefrom. Biological samples can also include sections of tissues, such as frozen sections or formalin fixed sections taken for histological purposes.

As used herein, term “comprising”, which is synonymous with “including,” “containing”, or “characterized by”, is inclusive or open-ended and does not exclude additional, unrecited elements and/or method steps. “Comprising” is a term of art used in claim language which means that the named elements are present, but other elements can be added and still form a composition or method within the scope of the presently disclosed subject matter. By way of example and not limitation, a pharmaceutical composition comprising a particular active agent and a pharmaceutically acceptable carrier can also contain other components including, but not limited to other active agents, other carriers and excipients, and any other molecule that might be appropriate for inclusion in the pharmaceutical composition without any limitation.

As used herein, the phrase “consisting of” excludes any element, step, or ingredient that is not particularly recited in the claim. When the phrase “consists of” appears in a clause of the body of a claim, rather than immediately following the preamble, it limits only the element set forth in that clause; other elements are not excluded from the claim as a whole. By way of example and not limitation, a pharmaceutical composition consisting of an active agent and a pharmaceutically acceptable carrier contains no other components besides the particular active agent and the pharmaceutically acceptable carrier. It is understood that any molecule that is below a reasonable level of detection is considered to be absent.

As used herein, the phrase “consisting essentially of” limits the scope of a claim to the specified materials or steps, plus those that do not materially affect the basic and novel characteristic(s) of the claimed subject matter. By way of example and not limitation, a pharmaceutical composition consisting essentially of an active agent and a pharmaceutically acceptable carrier contains active agent and the pharmaceutically acceptable carrier, but can also include any additional elements that might be present but that do not materially affect the biological functions of the composition in vitro or in vivo.

With respect to the terms “comprising”, “consisting essentially of”, and “consisting of”, where one of these three terms is used herein, the presently disclosed and claimed subject matter encompasses the use of either of the other two terms. For example, “comprising” is a transitional term that is broader than both “consisting essentially of” and “consisting of”, and thus the term “comprising” implicitly encompasses both “consisting essentially of” and “consisting of”. Likewise, the transitional phrase “consisting essentially of” is broader than “consisting of”, and thus the phrase “consisting essentially of” implicitly encompasses “consisting of”.

The term “subject” as used herein refers to a member of any invertebrate or vertebrate species. Accordingly, the term “subject” is intended to encompass any member of the Kingdom Animalia including, but not limited to the phylum Chordata (i.e., members of Classes Osteichythyes (bony fish), Amphibia (amphibians), Reptilia (reptiles), Ayes (birds), and Mammalia (mammals)), and all Orders and Families encompassed therein. In some embodiments, a subject is a human.

Similarly, all genes, gene names, gene products, and other products disclosed herein are intended to correspond to orthologs or other similar products from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates. Thus, for example, any genes specifically mentioned herein and for which Accession Nos. for various exemplary gene products disclosed in the GENBANK® biosequence database, are intended to encompass homologous and variant genes and gene products from humans and other animals including, but not limited to other mammals.

The methods of the presently disclosed subject matter are particularly useful for warm-blooded vertebrates. Thus, the presently disclosed subject matter concerns mammals and birds. More particularly contemplated is the isolation, manipulation, and use of stem cells from mammals such as humans and other primates, as well as those mammals of importance due to being endangered (such as Siberian tigers), of economic importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), rodents (such as mice, rats, and rabbits), marsupials, and horses. Also provided is the use of the disclosed methods and compositions on birds, including those kinds of birds that are endangered, kept in zoos, as well as fowl, and more particularly domesticated fowl, e.g., poultry, such as turkeys, chickens, ducks, geese, guinea fowl, and the like, as they are also of economic importance to humans. Thus, also contemplated is the isolation, manipulation, and use of stem cells from livestock, including but not limited to domesticated swine (pigs and hogs), ruminants, horses, poultry, and the like.

As used herein, the phrase “substantially” refers to a condition wherein in some embodiments no more than 50%, in some embodiments no more than 40%, in some embodiments no more than 30%, in some embodiments no more than 25%, in some embodiments no more than 20%, in some embodiments no more than 15%, in some embodiments no more than 10%, in some embodiments no more than 9%, in some embodiments no more than 8%, in some embodiments no more than 7%, in some embodiments no more than 6%, in some embodiments no more than 5%, in some embodiments no more than 4%, in some embodiments no more than 3%, in some embodiments no more than 2%, in some embodiments no more than 1%, and in some embodiments no more than 0% of the components of a collection of entities does not have a given characteristic.

The terms “additional therapeutically active compound” or “additional therapeutic agent”, as used in the context of the presently disclosed subject matter, refer to the use or administration of a compound for an additional therapeutic use for a particular injury, disease, or disorder being treated. Such a compound, for example, could include one being used to treat an unrelated disease or disorder, or a disease or disorder which is not responsive to the primary treatment for the injury, disease or disorder being treated. Diseases and disorders being treated by the additional therapeutically active agent include, for example, hypertension and diabetes. The additional compounds can also be used to treat symptoms associated with the injury, disease, or disorder, including, but not limited to, pain and inflammation.

The term “adult” as used herein, is meant to refer to any non-embryonic or non-juvenile subject.

As used herein, an “agonist” is a composition of matter which, when administered to a mammal such as a human, enhances or extends a biological activity attributable to the level or presence of a target compound or molecule of interest in the subject.

A disease or disorder is “alleviated” if the severity of a symptom of the disease, condition, or disorder, or the frequency with which such a symptom is experienced by a subject, or both, are reduced.

As used herein, amino acids are represented by the full name thereof, by the three letter code corresponding thereto, or by the one-letter code corresponding thereto, as indicated in Table 1:

TABLE 1
Amino Acid Codes and Functionally Equivalent Codons

| | 3-Letter | 1-Letter | |
|---|---|---|---|
| Full Name | Code | Code | Functionally Equivalent Codons |
| Aspartic Acid | Asp | D | GAC; GAU |
| Glutamic Acid | Glu | E | GAA; GAG |
| Lysine | Lys | K | AAA; AAG |
| Arginine | Arg | R | AGA; AGG; CGA; CGC; CGG; |
| | | | CGU |
| Histidine | His | H | CAC; CAU |
| Tyrosine | Tyr | Y | UAC; UAU |
| Cysteine | Cys | C | UGC; UGU |
| Asparagine | Asn | N | AAC; AAU |
| Glutamine | Gln | Q | CAA; CAG |
| Serine | Ser | S | ACG; AGU; UCA; UCC; UCG; |
| | | | UCU |
| Threonine | Thr | T | ACA; ACC; ACG; ACU |
| Glycine | Gly | G | GGA; GGC; GGG; GGU |
| Alanine | Ala | A | GCA; GCC; GCG; GCU |
| Valine | Val | V | GUA; GUC; GUG; GUU |
| Leucine | Leu | L | UUA; UUG; CUA; CUC; CUG; |
| | | | CUU |
| Isoleucine | Ile | I | AUA; AUC; AUU |
| Methionine | Met | M | AUG |
| Proline | Pro | P | CCA; CCC; CCG; CCU |
| Phenylalanine | Phe | F | UUC; UUU |
| Tryptophan | Trp | W | UGG |

The expression “amino acid” as used herein is meant to include both natural and synthetic amino acids, and both D and L amino acids. “Standard amino acid” means any of the twenty standard L-amino acids commonly found in naturally occurring peptides. “Nonstandard amino acid residue” means any amino acid, other than the standard amino acids, regardless of whether it is prepared synthetically or derived from a natural source. As used herein, “synthetic amino acid” also encompasses chemically modified amino acids, including but not limited to salts, amino acid derivatives (such as amides), and substitutions. Amino acids contained within the peptides of the presently disclosed subject matter, and particularly at the carboxy- or amino-terminus, can be modified by methylation, amidation, acetylation or substitution with other chemical groups which can change the peptide's circulating half-life without adversely affecting their activity. Additionally, a disulfide linkage may be present or absent in the peptides of the presently disclosed subject matter.

The term “amino acid” is used interchangeably with “amino acid residue,” and can refer to a free amino acid or to an amino acid residue of a peptide. It will be apparent from the context in which the term is used whether it refers to a free amino acid or a residue of a peptide.

Amino acids can be classified into seven groups on the basis of the side chain R: (1) aliphatic side chains, (2) side chains containing a hydroxylic (OH) group, (3) side chains containing sulfur atoms, (4) side chains containing an acidic or amide group, (5) side chains containing a basic group, (6) side chains containing an aromatic ring, and (7) proline, an imino acid in which the side chain is fused to the amino group.

Amino acids have the following general structure:

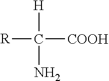

The nomenclature used to describe the peptide compounds of the presently disclosed subject matter follows the conventional practice wherein the amino group is presented to the left and the carboxy group to the right of each amino acid residue. In the formulae representing selected specific embodiments of the presently disclosed subject matter, the amino- and carboxy-terminal groups, although not specifically shown, will be understood to be in the form they would assume at physiologic pH values, unless otherwise specified.

The term “basic” or “positively charged” amino acid, as used herein, refers to amino acids in which the R groups have a net positive charge at pH 7.0, and include, but are not limited to, the standard amino acids lysine, arginine, and histidine.

As used herein, an “analog” of a chemical compound is a compound that, by way of example, resembles another in structure but is not necessarily an isomer (e.g., 5-fluorouracil is an analog of thymine).

An “antagonist” is a composition of matter which when administered to a mammal such as a human, inhibits a biological activity attributable to the level or presence of a compound or molecule of interest in the subject.

The term “antibody”, as used herein, refers to an immunoglobulin molecule which is able to specifically or selectively bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the presently disclosed subject matter can exist in a variety of forms. The term “antibody” refers to polyclonal and monoclonal antibodies and derivatives thereof (including chimeric, synthesized, humanized and human antibodies), including an entire immunoglobulin or antibody or any functional fragment of an immunoglobulin molecule which binds to the target antigen and or combinations thereof. Examples of such functional entities include complete antibody molecules, antibody fragments, such as Fᵥ, single chain Fᵥ, complementarity determining regions (CDRs), V_{L} (light chain variable region), V_{H} (heavy chain variable region), Fab, F(ab′)₂ and any combination of those or any other functional portion of an immunoglobulin peptide capable of binding to target antigen.

Antibodies exist, e.g., as intact immunoglobulins or as a number of well characterized fragments produced by digestion with various peptidases. Thus, for example, pepsin digests an antibody below the disulfide linkages in the hinge region to produce F(ab′)₂ a dimer of Fab which itself is a light chain joined to V_{H}-C_{H1} by a disulfide bond. The F(ab′)₂ can be reduced under mild conditions to break the disulfide linkage in the hinge region, thereby converting the F(ab′)₂ dimer into an Fab₁ monomer. The Fab₁ monomer is essentially a Fab with part of the hinge region (see Paul, 1993). While various antibody fragments are defined in terms of the digestion of an intact antibody, one of skill will appreciate that such fragments can be synthesized de novo either chemically or by utilizing recombinant DNA methodology. Thus, the term antibody, as used herein, also includes antibody fragments either produced by the modification of whole antibodies or those synthesized de novo using recombinant DNA methodologies.

An “antibody heavy chain”, as used herein, refers to the larger of the two types of polypeptide chains present in all intact antibody molecules.

An “antibody light chain”, as used herein, refers to the smaller of the two types of polypeptide chains present in all intact antibody molecules.

The term “single chain antibody” refers to an antibody wherein the genetic information encoding the functional fragments of the antibody are located in a single contiguous length of DNA. For a thorough description of single chain antibodies, see Bird et al., 1988; Huston et al., 1988).

The term “humanized” refers to an antibody wherein the constant regions have at least about 80% or greater homology to human immunoglobulin. Additionally, some of the nonhuman, such as murine, variable region amino acid residues can be modified to contain amino acid residues of human origin. Humanized antibodies have been referred to as “reshaped” antibodies. Manipulation of the complementarity-determining regions (CDR) is a way of achieving humanized antibodies. See for example, U.S. Pat. Nos. 4,816,567; 5,482,856; 6,479,284; 6,677,436; 7,060,808; 7,906,625; 8,398,980; 8,436,150; 8,796,439; and 10,253,111; and U.S. Patent Application Publication Nos. 2003/0017534, 2018/0298087, 2018/0312588, 2018/0346564, and 2019/0151448, each of which is incorporated by reference in its entirety.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “antigen” as used herein is defined as a molecule that provokes an immune response. This immune response can involve either antibody production, or the activation of specific immunologically-competent cells, or both. An antigen can be derived from organisms, subunits of proteins/antigens, killed or inactivated whole cells or lysates.

The term “antimicrobial agents” as used herein refers to any naturally-occurring, synthetic, or semi-synthetic compound or composition or mixture thereof, which is safe for human or animal use as practiced in the methods of the presently disclosed subject matter, and is effective in killing or substantially inhibiting the growth of microbes. “Antimicrobial” as used herein, includes antibacterial, antifungal, and antiviral agents.

As used herein, the term “antisense oligonucleotide” or antisense nucleic acid means a nucleic acid polymer, at least a portion of which is complementary to a nucleic acid which is present in a normal cell or in an affected cell. “Antisense” refers particularly to the nucleic acid sequence of the non-coding strand of a double stranded DNA molecule encoding a protein, or to a sequence which is substantially homologous to the non-coding strand. As defined herein, an antisense sequence is complementary to the sequence of a double stranded DNA molecule encoding a protein. It is not necessary that the antisense sequence be complementary solely to the coding portion of the coding strand of the DNA molecule. The antisense sequence can be complementary to regulatory sequences specified on the coding strand of a DNA molecule encoding a protein, which regulatory sequences control expression of the coding sequences. The antisense oligonucleotides of the presently disclosed subject matter include, but are not limited to, phosphorothioate oligonucleotides and other modifications of oligonucleotides.

The term “autologous”, as used herein, refers to something that occurs naturally and normally in a certain type of tissue or in a specific structure of the body. In transplantation, it refers to a graft in which the donor and recipient areas are in the same individual, or to blood that the donor has previously donated and then receives back, usually during surgery.

The term “basal medium”, as used herein, refers to a minimum essential type of medium, such as Dulbecco's Modified Eagle's Medium, Ham's F12, Eagle's Medium, RPMI, ARB, etc., to which other ingredients can be added. The term does not exclude media which have been prepared or are intended for specific uses, but which upon modification can be used for other cell types, etc.

The term “biocompatible”, as used herein, refers to a material that does not elicit a substantial detrimental response in the host.

The term “biodegradable”, as used herein, means capable of being biologically decomposed. A biodegradable material differs from a non-biodegradable material in that a biodegradable material can be biologically decomposed into units which can be either removed from the biological system and/or chemically incorporated into the biological system.

The term “biological sample”, as used herein, refers to samples obtained from a living organism, including skin, hair, tissue, blood, plasma, cells, sweat, and urine.

The term “bioresorbable”, as used herein, refers to the ability of a material to be resorbed in vivo. “Full” resorption means that no significant extracellular fragments remain. The resorption process involves elimination of the original implant materials through the action of body fluids, enzymes, or cells. Resorbed calcium carbonate can, for example, be redeposited as bone mineral, or by being otherwise re-utilized within the body, or excreted. “Strongly bioresorbable”, as the term is used herein, means that at least 80% of the total mass of material implanted is resorbed within one year.

The phrases “cell culture medium”, “culture medium” (plural “media” in each case), and “medium formulation” refer to a nutritive solution for cultivating cells and may be used interchangeably.

A “conditioned medium” is one prepared by culturing a first population of cells or tissue in a medium, and then harvesting the medium. The conditioned medium (along with anything secreted into the medium by the cells) can then be used in any desired way, such as to treat a disease or disorder in a subject, or to support the growth or differentiation of a second population of cells.

As used herein, the term “conservative amino acid substitution” is defined herein as an amino acid exchange within one of the five groups summarized in the following Table 2.

TABLE 2
Conservative Amino Acid Substitutions

| Group | Characteristics | Amino Acids |
|---|---|---|
| A. | Small aliphatic, nonpolar | Ala, Ser, Thr, Pro, Gly |
| | or slightly polar residues | |
| B. | Polar, negatively charged | Asp, Asn, Glu, Gln |
| | residues and their amides | |
| C. | Polar, positively charged residues | His, Arg, Lys |
| D. | Large, aliphatic, nonpolar residues | Met Leu, Ile, Val, Cys |
| E. | Large, aromatic residues | Phe, Tyr, Trp |

A “control” cell, tissue, sample, or subject is a cell, tissue, sample, or subject of the same type as a test cell, tissue, sample, or subject. The control can, for example, be examined at precisely or nearly the same time the test cell, tissue, sample, or subject is examined. The control can also, for example, be examined at a time distant from the time at which the test cell, tissue, sample, or subject is examined, and the results of the examination of the control can be recorded so that the recorded results can be compared with results obtained by examination of a test cell, tissue, sample, or subject. The control can also be obtained from another source or similar source other than the test group or a test subject, where the test sample is obtained from a subject suspected of having a disease or disorder for which the test is being performed.

A “test” cell, tissue, sample, or subject is one being examined or treated.

A “pathoindicative” cell, tissue, or sample is one which, when present, is an indication that the animal in which the cell, tissue, or sample is located (or from which the tissue was obtained) is afflicted with a disease or disorder. By way of example, the presence of one or more breast cells in a lung tissue of an animal is an indication that the animal is afflicted with metastatic breast cancer.

A tissue “normally comprises” a cell if one or more of the cells are present in the tissue in an animal not afflicted with a disease or disorder.

A “compound”, as used herein, refers to any type of substance or agent that is commonly considered a drug, or a candidate for use as a drug, combinations, and mixtures of the above, as well as polypeptides and antibodies of the presently disclosed subject matter.

“Cytokine”, as used herein, refers to intercellular signaling molecules, the best known of which are involved in the regulation of mammalian somatic cells. A number of families of cytokines, both growth promoting and growth inhibitory in their effects, have been characterized including, for example, interleukins, interferons, and transforming growth factors. A number of other cytokines are known to those of skill in the art. The sources, characteristics, targets, and effector activities of these cytokines have been described.

“Chemokine”, as used herein, refers to an intercellular signaling molecule involved in the chemotaxis of white blood cells, such as T cells.

The term “delivery vehicle” refers to any kind of device or material, which can be used to deliver cells in vivo or can be added to a composition comprising cells administered to an animal. This includes, but is not limited to, implantable devices, aggregates of cells, matrix materials, gels, etc.

As used herein, a “derivative” of a compound refers to a chemical compound that can be produced from another compound of similar structure in one or more steps, as in replacement of H by an alkyl, acyl, or amino group.

The use of the word “detect” and its grammatical variants is meant to refer to measurement of the species without quantification, whereas use of the word “determine” or “measure” with their grammatical variants are meant to refer to measurement of the species with quantification. The terms “detect” and “identify” are used interchangeably herein.

As used herein, a “detectable marker” or a “reporter molecule” is an atom or a molecule that permits the specific detection of a compound comprising the marker in the presence of similar compounds without a marker. Detectable markers or reporter molecules include, e.g., radioactive isotopes, antigenic determinants, enzymes, nucleic acids available for hybridization, chromophores, fluorophores, chemiluminescent molecules, electrochemically detectable molecules, and molecules that provide for altered fluorescence-polarization or altered light-scattering.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, an “effective amount” means an amount sufficient to produce a selected effect. A “therapeutically effective amount” means an effective amount of an agent being used in treating or preventing a disease or disorder.

The term “epitope” as used herein is defined as small chemical groups on the antigen molecule that can elicit and react with an antibody. An antigen can have one or more epitopes. Most antigens have many epitopes; i.e., they are multivalent. In general, an epitope is roughly five amino acids or sugars in size. One skilled in the art understands that generally the overall three-dimensional structure, rather than the specific linear sequence of the molecule, is the main criterion of antigenic specificity.

A “fragment” or “segment” is a portion of an amino acid sequence, comprising at least one amino acid, or a portion of a nucleic acid sequence comprising at least one nucleotide. The terms “fragment” and “segment” are used interchangeably herein. As used herein, the term “fragment”, as applied to a protein or peptide, can ordinarily be at least about 3-15 amino acids in length, at least about 15-25 amino acids, at least about 25-50 amino acids in length, at least about 50-75 amino acids in length, at least about 75-100 amino acids in length, and greater than 100 amino acids in length.

As used herein, the term “fragment” as applied to a nucleic acid, may ordinarily be at least about 20 nucleotides in length, typically, at least about 50 nucleotides, more typically, from about 50 to about 100 nucleotides, in some embodiments, at least about 100 to about 200 nucleotides, in some embodiments, at least about 200 nucleotides to about 300 nucleotides, yet in some embodiments, at least about 300 to about 350, in some embodiments, at least about 350 nucleotides to about 500 nucleotides, yet in some embodiments, at least about 500 to about 600, in some embodiments, at least about 600 nucleotides to about 620 nucleotides, yet in some embodiments, at least about 620 to about 650, and most in some embodiments, the nucleic acid fragment will be greater than about 650 nucleotides in length.

As used herein, a “functional” molecule is a molecule in a form in which it exhibits a property or activity by which it is characterized.

As used herein, a “functional biological molecule” is a biological molecule in a form in which it exhibits a property by which it is characterized. A functional enzyme, for example, is one which exhibits the characteristic catalytic activity by which the enzyme is characterized.

The term “growth factor” as used herein means a bioactive molecule that promotes the proliferation of a cell or tissue. Growth factors useful in the presently disclosed subject matter include, but are not limited to, transforming growth factor-alpha (TGF-α), transforming growth factor-beta (TGF-β), platelet-derived growth factors including the AA, AB and BB isoforms (PDGF), fibroblast growth factors (FGF), including FGF acidic isoforms 1 and 2, FGF basic form 2, and FGF 4, 8, 9, and 10, nerve growth factors (NGF) including NGF 2.5s, NGF 7.0s, and beta NGF and neurotrophins, brain derived neurotrophic factor, cartilage derived factor, bone growth factors (BGF), basic fibroblast growth factor, insulin-like growth factor (IGF), vascular endothelial growth factor (VEGF), EG-VEGF, VEGF-related protein, Bv8, VEGF-E, granulocyte colony stimulating factor (G-CSF), insulin like growth factor (IGF) I and II, hepatocyte growth factor, glial neurotrophic growth factor, stem cell factor (SCF), keratinocyte growth factor (KGF), skeletal growth factor, bone matrix derived growth factors, and bone derived growth factors and mixtures thereof. Some growth factors may also promote differentiation of a cell or tissue. TGF, for example, may promote growth and/or differentiation of a cell or tissue.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 5′-ATTGCC-3′ and 5′-TATGGC-3′ share 50% homology.

As used herein, “homology” is used synonymously with “identity”.

The determination of percent identity between two nucleotide or amino acid sequences can be accomplished using a mathematical algorithm. For example, a mathematical algorithm useful for comparing two sequences is the algorithm of Karlin & Altschul (1990) Methods for assessing the statistical significance of molecular sequence features by using general scoring schemes. Proc Natl Acad Sci USA 87:2264-2268, modified as in Karlin & Altschul (1993) Applications and statistics for multiple high-scoring segments in molecular sequences. Proc Natl Acad Sci USA 90:5873-5877). This algorithm is incorporated into the NBLAST and XBLAST programs (see Altschul et al. (1990a) Basic local alignment search tool. J Mol Biol 215:403-410; Altschul et al. (1990b) Protein database searches for multiple alignments. Proc Natl Acad Sci USA 87:14:5509-5513, and can be accessed, for example at the National Center for Biotechnology Information (NCBI) world wide web site. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al. (1997) Gapped BLAST and PSI-BLAST: a new generation of protein database search programs. Nucleic Acids Res 25:3389-3402. Alternatively, PSI-Blast or PHI-Blast can be used to perform an iterated search which detects distant relationships between molecules (Id.) and relationships between molecules which share a common pattern. When utilizing BLAST, Gapped BLAST, PSI-Blast, and PHI-Blast programs, the default parameters of the respective programs (e.g., XBLAST and NBLAST) can be used.

The percent identity between two sequences can be determined using techniques similar to those described above, with or without allowing gaps. In calculating percent identity, typically exact matches are counted.

As used herein, the term “hybridization” is used in reference to the pairing of complementary nucleic acids. Hybridization and the strength of hybridization (i.e., the strength of the association between the nucleic acids) is impacted by such factors as the degree of complementarity between the nucleic acids, stringency of the conditions involved, the length of the formed hybrid, and the G:C ratio within the nucleic acids.

The term “ingredient” refers to any compound, whether of chemical or biological origin, that can be used in cell culture media to maintain or promote the proliferation, survival, or differentiation of cells. The terms “component”, “nutrient”, “supplement”, and ingredient” can be used interchangeably and are all meant to refer to such compounds. Typical non-limiting ingredients that are used in cell culture media include amino acids, salts, metals, sugars, lipids, nucleic acids, hormones, vitamins, fatty acids, proteins, and the like. Other ingredients that promote or maintain cultivation of cells ex vivo can be selected by those of skill in the art, in accordance with the particular need.

The term “inhibit”, as used herein, refers to the ability of a compound, agent, or method to reduce or impede a described function, level, activity, rate, etc., based on the context in which the term “inhibit” is used. In some embodiments, inhibition is by at least 10%, in some embodiments by at least 25%, in some embodiments by at least 50%, and in some embodiments, the function is inhibited by at least 75%. The term “inhibit” is used interchangeably with “reduce” and “block”.

The term “inhibitor” as used herein, refers to any compound or agent, the application of which results in the inhibition of a process or function of interest, including, but not limited to, differentiation and activity. Inhibition can be inferred if there is a reduction in the activity or function of interest.

As used herein “injecting or applying” includes administration of a compound or composition of the presently disclosed subject matter by any number of routes and approaches including, but not limited to, topical, oral, buccal, intravenous, intratumoral, intramuscular, intra-arterial, intramedullary, intrathecal, intraventricular, transdermal, subcutaneous, intraperitoneal, intranasal, enteral, topical, sublingual, vaginal, ophthalmic, pulmonary, or rectal means.

As used herein, “injury” generally refers to damage, harm, or hurt; usually applied to damage inflicted on the body by an external force.

As used herein, an “instructional material” includes a publication, a recording, a diagram, or any other medium of expression, which can be used to communicate the usefulness of the composition of the presently disclosed subject matter in the kit for effecting alleviation of the various diseases or disorders recited herein. Optionally, or alternately, the instructional material may describe one or more methods of alleviating the diseases or disorders in a cell or a tissue of a mammal. The instructional material of the kit of the presently disclosed subject matter may, for example, be affixed to a container, which contains the identified compound presently disclosed subject matter, or be shipped together to with a container, which contains the identified compound. Alternatively, the instructional material can be shipped separately from the container with the intention that the instructional material and the compound be used cooperatively by the recipient.

Used interchangeably herein are the terms “isolate” and “select”.

The terms “isolate”, “isolated”, “isolating”, and grammatical variations thereof when used in reference to TITE or cells, refers to a single TITE or cell of interest, or a population of TITE or cells of interest, at least partially isolated from other cell types or other cellular material with which it occurs in a culture or a tissue of origin. A sample is “substantially pure” when it is in some embodiments at least 60%, in some embodiments at least 75%, in some embodiments at least 90%, and, in certain cases, in some embodiments at least 99% free of cells or other cellular material other than TITE or cells of interest. Purity can be measured by any appropriate method, such as but not limited to those presented in the EXAMPLES.

An “isolated nucleic acid” refers to a nucleic acid segment or fragment, which has been separated from sequences, which flank it in a naturally occurring state, e.g., a DNA fragment that has been removed from the sequences, which are normally adjacent to the fragment, e.g., the sequences adjacent to the fragment in a genome in which it naturally occurs. The term also applies to nucleic acids, which have been substantially purified, from other components, which naturally accompany the nucleic acid, e.g., RNA or DNA, or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA, which is part of a hybrid gene encoding additional polypeptide sequence.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

As used herein, a “ligand” is a compound that specifically binds to a target compound. A ligand (e.g., an antibody) “specifically binds to” or “is specifically immunoreactive with” a compound when the ligand functions in a binding reaction which is determinative of the presence of the compound in a sample of heterogeneous compounds. Thus, under designated assay (e.g., immunoassay) conditions, the ligand binds preferentially to a particular compound and does not bind to a significant extent to other compounds present in the sample. For example, an antibody specifically binds under immunoassay conditions to an antigen bearing an epitope against which the antibody was raised. A variety of immunoassay formats may be used to select antibodies specifically immunoreactive with a particular antigen. For example, solid-phase ELISA immunoassays are routinely used to select monoclonal antibodies specifically immunoreactive with an antigen. See Harlow & Lane, 1988 for a description of immunoassay formats and conditions that can be used to determine specific immunoreactivity.

A “receptor” is a compound that specifically or selectively binds to a ligand.

As used herein, the term “linkage” refers to a connection between two groups. The connection can be either covalent or non-covalent, including but not limited to ionic bonds, hydrogen bonding, and hydrophobic/hydrophilic interactions.

As used herein, the term “linker” refers to a molecule or bivalent group derived therefrom that joins two other molecules covalently or noncovalently, e.g., through ionic or hydrogen bonds or van der Waals interactions.

The term “measuring the level of expression” or “determining the level of expression” as used herein refers to any measure or assay which can be used to correlate the results of the assay with the level of expression of a gene or protein of interest. Such assays include measuring the level of mRNA, protein levels, etc. and can be performed by assays such as northern and western blot analyses, binding assays, immunoblots, etc. The level of expression can include rates of expression and can be measured in terms of the actual amount of an mRNA or protein present. Such assays are coupled with processes or systems to store and process information and to help quantify levels, signals, etc. and to digitize the information for use in comparing levels.

Micro-RNAs are generally about 16-25 nucleotides in length. In some embodiments, miRNAs are RNA molecules of 22 nucleotides or less in length. These molecules have been found to be highly involved in the pathology of several types of cancer. Although the miRNA molecules are generally found to be stable when associated with blood serum and its components after EDTA treatment, introduction of locked nucleic acids (LNAs) to the miRNAs via PCR further increases stability of the miRNAs. LNAs are a class of nucleic acid analogues in which the ribose ring is “locked” by a methylene bridge connecting the 2′-O atom and the 4′-C atom of the ribose ring, which increases the molecule's affinity for other molecules. miRNAs are species of small non-coding single-stranded regulatory RNAs that interact with the 3′-untranslated region (3′-UTR) of target mRNA molecules through partial sequence homology. They participate in regulatory networks as controlling elements that direct comprehensive gene expression. Bioinformatics analysis has predicted that a single miRNA can regulate hundreds of target genes, contributing to the combinational and subtle regulation of numerous genetic pathways.

The term “modulate”, as used herein, refers to changing the level of an activity, function, or process. The term “modulate” encompasses both inhibiting and stimulating an activity, function, or process. The term “modulate” is used interchangeably with the term “regulate” herein.

The term “nucleic acid” typically refers to large polynucleotides. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine, and uracil).

As used herein, the term “nucleic acid” encompasses RNA as well as single and double stranded DNA and cDNA. Furthermore, the terms, “nucleic acid”, “DNA”, “RNA” and similar terms also include nucleic acid analogs, i.e. analogs having other than a phosphodiester backbone. For example, the so called “peptide nucleic acids”, which are known in the art and have peptide bonds instead of phosphodiester bonds in the backbone, are considered within the scope of the presently disclosed subject matter. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine, and uracil). Conventional notation is used herein to describe polynucleotide sequences: the left-hand end of a single-stranded polynucleotide sequence is the 5′-end; the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences”.

The term “nucleic acid construct”, as used herein, encompasses DNA and RNA sequences encoding the particular gene or gene fragment desired, whether obtained by genomic or synthetic methods.

Unless otherwise specified, a “nucleotide sequence encoding an amino acid sequence” includes all nucleotide sequences that are degenerate versions of each other and that encode the same amino acid sequence. Nucleotide sequences that encode proteins and

RNA may include introns.

The term “oligonucleotide” typically refers to short polynucleotides, generally, no greater than about 50 nucleotides. It will be understood that when a nucleotide sequence is represented by a DNA sequence (i.e., A, T, G, C), this also includes an RNA sequence (i.e., A, U, G, C) in which “U” replaces “T”.

By describing two polynucleotides as “operably linked” is meant that a single-stranded or double-stranded nucleic acid moiety comprises the two polynucleotides arranged within the nucleic acid moiety in such a manner that at least one of the two polynucleotides is able to exert a physiological effect by which it is characterized upon the other. By way of example, a promoter operably linked to the coding region of a gene is able to promote transcription of the coding region.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, intratumoral, and kidney dialytic infusion techniques.

“Permeation enhancement” and “permeation enhancers” as used herein relate to the process and added materials which bring about an increase in the permeability of skin to a poorly skin permeating pharmacologically active agent, i.e., so as to increase the rate at which the drug permeates through the skin and enters the bloodstream. “Permeation enhancer” is used interchangeably with “penetration enhancer”.

The term “pharmaceutical composition” shall mean a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

As used herein, the term “pharmaceutically-acceptable carrier” means a chemical composition with which an appropriate compound or derivative can be combined and which, following the combination, can be used to administer the appropriate compound to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

“Plurality” means at least two.

A “polynucleotide” means a single strand or parallel and anti-parallel strands of a nucleic acid. Thus, a polynucleotide may be either a single-stranded or a double-stranded nucleic acid.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof.

“Synthetic peptides or polypeptides” means a non-naturally occurring peptide or polypeptide. Synthetic peptides or polypeptides can be synthesized, for example, using an automated polypeptide synthesizer. Various solid phase peptide synthesis methods are known to those of skill in the art.

The term “prevent”, as used herein, means to stop something from happening, or taking advance measures against something possible or probable from happening. In the context of medicine, “prevention” generally refers to action taken to decrease the chance of getting a disease or condition.

“Primer” refers to a polynucleotide that is capable of specifically hybridizing to a designated polynucleotide template and providing a point of initiation for synthesis of a complementary polynucleotide. Such synthesis occurs when the polynucleotide primer is placed under conditions in which synthesis is induced, i.e., in the presence of nucleotides, a complementary polynucleotide template, and an agent for polymerization such as DNA polymerase. A primer is typically single-stranded, but may be double-stranded. Primers are typically deoxyribonucleic acids, but a wide variety of synthetic and naturally occurring primers are useful for many applications. A primer is complementary to the template to which it is designed to hybridize to serve as a site for the initiation of synthesis, but need not reflect the exact sequence of the template. In such a case, specific hybridization of the primer to the template depends on the stringency of the hybridization conditions. Primers can be labeled with, e.g., chromogenic, radioactive, or fluorescent moieties and used as detectable moieties.

A “prophylactic” treatment is a treatment administered to a subject who does not exhibit signs of a disease or injury or exhibits only early signs of the disease or injury for the purpose of decreasing the risk of developing pathology associated with the disease or injury.

As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a promoter which drives expression of a gene to which it is operably linked, in a constant manner in a cell. By way of example, promoters which drive expression of cellular housekeeping genes are considered to be constitutive promoters.

An “inducible” promoter is a nucleotide sequence which, when operably linked with a polynucleotide which encodes or specifies a gene product, causes the gene product to be produced in a living cell substantially only when an inducer which corresponds to the promoter is present in the cell.

A “tissue-specific” promoter is a nucleotide sequence which, when operably linked with a polynucleotide which encodes or specifies a gene product, causes the gene product to be produced in a living cell substantially only if the cell is a cell of the tissue type corresponding to the promoter.

As used herein, “protecting group” with respect to a terminal amino group refers to a terminal amino group of a peptide, which terminal amino group is coupled with any of various amino-terminal protecting groups traditionally employed in peptide synthesis. Such protecting groups include, for example, acyl protecting groups such as formyl, acetyl, benzoyl, trifluoroacetyl, succinyl, and methoxysuccinyl; aromatic urethane protecting groups such as benzyloxycarbonyl; and aliphatic urethane protecting groups, for example, tert-butoxycarbonyl or adamantyloxycarbonyl. See Gross & Mienhofer, 1981 for suitable protecting groups.

As used herein, “protecting group” with respect to a terminal carboxy group refers to a terminal carboxyl group of a peptide, which terminal carboxyl group is coupled with any of various carboxyl-terminal protecting groups. Such protecting groups include, for example, tert-butyl, benzyl, or other acceptable groups linked to the terminal carboxyl group through an ester or ether bond.

The term “protein” typically refers to large polypeptides. Conventional notation is used herein to portray polypeptide sequences: the left-hand end of a polypeptide sequence is the amino-terminus; the right-hand end of a polypeptide sequence is the carboxyl-terminus.

The term “protein regulatory pathway”, as used herein, refers to both the upstream regulatory pathway which regulates a protein, as well as the downstream events which that protein regulates. Such regulation includes, but is not limited to, transcription, translation, levels, activity, posttranslational modification, and function of the protein of interest, as well as the downstream events which the protein regulates.

The terms “protein pathway” and “protein regulatory pathway” are used interchangeably herein.

As used herein, the term “purified” and like terms relate to an enrichment of a molecule or compound (e.g., TITE) relative to other components normally associated with the molecule or compound in a native environment. The term “purified” does not necessarily indicate that complete purity of the particular molecule has been achieved during the process. A “highly purified” compound as used herein refers to a compound that is greater than 90% pure.

“Recombinant polynucleotide” refers to a polynucleotide having sequences that are not naturally joined together. An amplified or assembled recombinant polynucleotide may be included in a suitable vector, and the vector can be used to transform a suitable host cell.

A recombinant polynucleotide can serve a non-coding function (e.g., promoter, origin of replication, ribosome-binding site, etc.), as well.

A host cell that comprises a recombinant polynucleotide is referred to as a “recombinant host cell”. A gene which is expressed in a recombinant host cell wherein the gene comprises a recombinant polynucleotide, produces a “recombinant polypeptide”.

A “recombinant polypeptide” is one which is produced upon expression of a recombinant polynucleotide.

The term “regulate” refers to either stimulating or inhibiting a function or activity of interest.

As used herein, term “regulatory elements” is used interchangeably with “regulatory sequences” and refers to promoters, enhancers, and other expression control elements, or any combination of such elements.

A “reversibly implantable” device is one which can be inserted (e.g., surgically or by insertion into a natural orifice of the animal) into the body of an animal and thereafter removed without great harm to the health of the animal.

A “sample”, as used herein, refers in some embodiments to a biological sample from a subject, including, but not limited to, normal tissue samples, diseased tissue samples, biopsies, blood, saliva, feces, semen, tears, and urine. A sample can also be any other source of material obtained from a subject which contains cells, tissues, or fluid of interest. A sample can also be obtained from cell or tissue culture.

A “significant detectable level” is an amount of contaminate that would be visible in the presented data and would need to be addressed/explained during analysis of the forensic evidence.

By the term “signal sequence” is meant a polynucleotide sequence which encodes a peptide that directs the path a polypeptide takes within a cell, i.e., it directs the cellular processing of a polypeptide in a cell, including, but not limited to, eventual secretion of a polypeptide from a cell. A signal sequence is a sequence of amino acids which are typically, but not exclusively, found at the amino terminus of a polypeptide which targets the synthesis of the polypeptide to the endoplasmic reticulum. In some instances, the signal peptide is proteolytically removed from the polypeptide and is thus absent from the mature protein.

By “small interfering RNAs (siRNAs)” is meant, inter alia, an isolated dsRNA molecule comprised of both a sense and an anti-sense strand. In some embodiments, it is greater than 10 nucleotides in length. siRNA also refers to a single transcript which has both the sense and complementary antisense sequences from the target gene, e.g., a hairpin. siRNA further includes any form of dsRNA (proteolytically cleaved products of larger dsRNA, partially purified RNA, essentially pure RNA, synthetic RNA, recombinantly produced RNA) as well as altered RNA that differs from naturally occurring RNA by the addition, deletion, substitution, and/or alteration of one or more nucleotides.

As used herein, the term “secondary antibody” refers to an antibody that binds to the constant region of another antibody (the primary antibody).

As used herein, the term “single chain variable fragment” (scFv) refers to a single chain antibody fragment comprised of a heavy and light chain linked by a peptide linker. In some cases, scFv are expressed on the surface of an engineered cell, for the purpose of selecting particular scFv that bind to an antigen of interest.

The terms “solid support”, “surface” and “substrate” are used interchangeably and refer to a structural unit of any size, where said structural unit or substrate has a surface suitable for immobilization of molecular structure or modification of said structure and said substrate is made of a material such as, but not limited to, metal, metal films, glass, fused silica, synthetic polymers, and membranes.

By the term “specifically binds”, as used herein, is meant a molecule which recognizes and binds a specific molecule, but does not substantially recognize or bind other molecules in a sample, or it means binding between two or more molecules as in part of a cellular regulatory process, where said molecules do not substantially recognize or bind other molecules in a sample.

The term “standard”, as used herein, refers to something used for comparison. For example, it can be a known standard agent or compound which is administered and used for comparing results when administering a test compound, or it can be a standard parameter or function which is measured to obtain a control value when measuring an effect of an agent or compound on a parameter or function. “Standard” can also refer to an “internal standard”, such as an agent or compound which is added at known amounts to a sample and which is useful in determining such things as purification or recovery rates when a sample is processed or subjected to purification or extraction procedures before a marker of interest is measured. Internal standards are often but are not always limited to, a purified marker of interest which has been labeled, such as with a radioactive isotope, allowing it to be distinguished from an endogenous substance in a sample.

The term “stimulate” as used herein, means to induce or increase an activity or function level such that it is higher relative to a control value. The stimulation can be via direct or indirect mechanisms. In some embodiments, the activity or function is stimulated by at least 10% compared to a control value, in some embodiments by at least 25%, and in some embodiments by at least 50%. The term “stimulator” as used herein, refers to any composition, compound or agent, the application of which results in the stimulation of a process or function of interest.

A “subject” of diagnosis or treatment is an animal, including a human. It also includes pets and livestock.

As used herein, a “subject in need thereof” is a patient, animal, mammal, or human, who will benefit from a method or compositions of the presently disclosed subject matter.

As used herein, “substantially homologous amino acid sequences” includes those amino acid sequences which have at least about 95% homology, in some embodiments at least about 96% homology, more in some embodiments at least about 97% homology, in some embodiments at least about 98% homology, and most in some embodiments at least about 99% or more homology to an amino acid sequence of a reference sequence. Amino acid sequence similarity or identity can be computed by using the BLASTP and TBLASTN programs which employ the BLAST (basic local alignment search tool) 2.0.14 algorithm. The default settings used for these programs are suitable for identifying substantially similar amino acid sequences for purposes of the presently disclosed subject matter.

“Substantially homologous nucleic acid sequence” means a nucleic acid sequence corresponding to a reference nucleic acid sequence wherein the corresponding sequence encodes a peptide having substantially the same structure and function as the peptide encoded by the reference nucleic acid sequence; e.g., where only changes in amino acids not significantly affecting the peptide function occur. In some embodiments, the substantially identical nucleic acid sequence encodes the peptide encoded by the reference nucleic acid sequence. The percentage of identity between the substantially similar nucleic acid sequence and the reference nucleic acid sequence is at least about 50%, 65%, 75%, 85%, 95%, 99% or more. Substantial identity of nucleic acid sequences can be determined by comparing the sequence identity of two sequences, for example by physical/chemical methods (i.e., hybridization) or by sequence alignment via computer algorithm. Suitable nucleic acid hybridization conditions to determine if a nucleotide sequence is substantially similar to a reference nucleotide sequence are: 7% sodium dodecyl sulfate SDS, 0.5 M NaPO4, 1 mM EDTA at 50° C. with washing in 2× standard saline citrate (SSC), 0.1% SDS at 50° C.; in some embodiments in 7% (SDS), 0.5 M NaPO4, 1 mM EDTA at 50° C. with washing in 1×SSC, 0.1% SDS at 50° C.; in some embodiments 7% SDS, 0.5 M NaPO4, 1 mM EDTA at 50° C. with washing in 0.5×SSC, 0.1% SDS at 50° C.; and more in some embodiments in 7% SDS, 0.5 M NaPO4, 1 mM EDTA at 50° C. with washing in 0.1×SSC, 0.1% SDS at 65° C. Suitable computer algorithms to determine substantial similarity between two nucleic acid sequences include, GCS program package, and the BLASTN or FASTA programs (Altschul et al., 1990a; Altschul et al., 1990b; Altschul et al., 1997). The default settings provided with these programs are suitable for determining substantial similarity of nucleic acid sequences for purposes of the presently disclosed subject matter.

The term “substantially pure” describes a compound, molecule, or the like, e.g., a TITE, which has been separated from components which naturally accompany it. Typically, a compound is substantially pure when at least 10%, more in some embodiments at least 20%, more in some embodiments at least 50%, more in some embodiments at least 60%, more in some embodiments at least 75%, more in some embodiments at least 90%, and most in some embodiments at least 99% of the total material (by volume, by wet or dry weight, or by mole percent or mole fraction) in a sample is the compound of interest. Purity can be measured by any appropriate method, e.g., those disclosed in the EXAMPLES, or in the case of polypeptides by column chromatography, gel electrophoresis, or HPLC analysis. A compound, e.g., a protein, is also substantially purified when it is essentially free of naturally associated components or when it is separated from the native contaminants which accompany it in its natural state.

A “surface active agent” or “surfactant” is a substance that has the ability to reduce the surface tension of materials and enable penetration into and through materials.

The term “symptom”, as used herein, refers to any morbid phenomenon or departure from the normal in structure, function, or sensation, experienced by the patient and indicative of disease. In contrast, a “sign” is objective evidence of disease. For example, a bloody nose is a sign. It is evident to the patient, doctor, nurse, and other observers.

A “therapeutic” treatment is a treatment administered to a subject who exhibits signs of pathology for the purpose of diminishing or eliminating those signs.

A “therapeutically effective amount” of a compound is that amount of compound which is sufficient to provide a beneficial effect to the subject to which the compound is administered.

“Tissue” means (1) a group of similar cell united perform a specific function; (2) a part of an organism consisting of an aggregate of cells having a similar structure and function; or (3) a grouping of cells that are similarly characterized by their structure and function, such as muscle or nerve tissue.

The term “topical application”, as used herein, refers to administration to a surface, such as the skin. This term is used interchangeably with “cutaneous application” in the case of skin. A “topical application” is a “direct application”.

By “transdermal” delivery is meant delivery by passage of a drug through the skin or mucosal tissue and into the bloodstream. Transdermal also refers to the skin as a portal for the administration of drugs or compounds by topical application of the drug or compound thereto. “Transdermal” is used interchangeably with “percutaneous”.

The term “transfection” is used interchangeably with the terms “gene transfer”, “transformation”, and “transduction”, and means the intracellular introduction of a polynucleotide. “Transfection efficiency” refers to the relative amount of the transgene taken up by the cells subjected to transfection. In practice, transfection efficiency is estimated by the amount of the reporter gene product expressed following the transfection procedure.

As used herein, the term “transgene” means an exogenous nucleic acid sequence comprising a nucleic acid which encodes a promoter/regulatory sequence operably linked to nucleic acid which encodes an amino acid sequence, which exogenous nucleic acid is encoded by a transgenic mammal.

As used herein, the term “treating” may include prophylaxis of the specific injury, disease, disorder, or condition, or alleviation of the symptoms associated with a specific injury, disease, disorder, or condition and/or preventing or eliminating said symptoms. A “prophylactic” treatment is a treatment administered to a subject who does not exhibit signs of a disease or exhibits only early signs of the disease for the purpose of decreasing the risk of developing pathology associated with the disease. “Treating” is used interchangeably with “treatment” herein.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer or delivery of nucleic acid to cells, such as, for example, polylysine compounds, liposomes, and the like. Examples of viral vectors include, but are not limited to, adenoviral vectors, adeno-associated virus vectors, retroviral vectors, recombinant viral vectors, and the like. Examples of non-viral vectors include, but are not limited to, liposomes, polyamine derivatives of DNA and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses that incorporate the recombinant polynucleotide.

The terminology used herein is for the purpose of describing the particular versions or embodiments only, and is not intended to limit the scope of the presently disclosed subject matter. All publications mentioned herein are incorporated by reference in their entirety.

### III. Representative Embodiments

III.A. Generally

Adoptive transfer of Bispecific antibody Armed activated T cells (BATs) shows promising anti-tumor activity in solid tumors (1-4). This strategy targets tumor cells using activated T cells (ATC) armed with bispecific antibodies (BiAb), of which one antibody binding domain binds to T cells and a second antibody binding domain binds the tumor antigen on the tumor cells. The cytotoxic activity occurs upon engagement of ATC with tumor cells via the bispecific antibody bridge that stimulates bispecific antibodies armed activated T cells (BATs) to release not only the lytic and cytotoxic molecules (perforin/granzyme) but also to release cytokines, chemokines and other signaling molecules extracellularly [5].

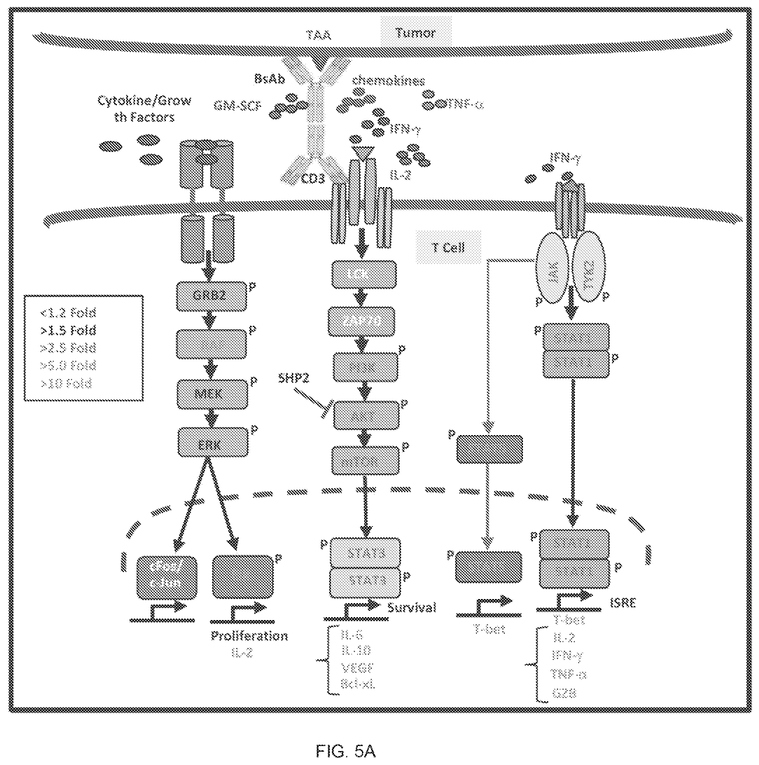

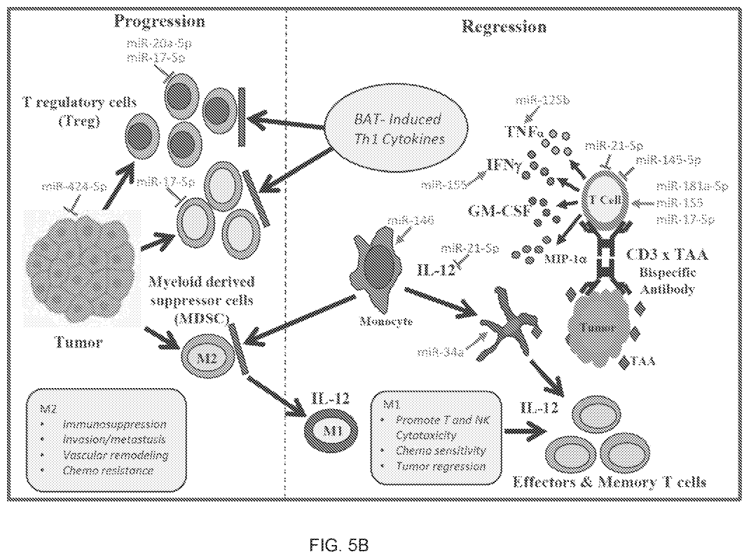

In accordance with aspects of the presently disclosed subject matter, it was observed that the release of BAT Induced Tumor-Targeting Effectors (TITE) by the complex interaction of T cells, bispecific antibody, and tumor cells serves as a potent anti-tumor and immune activating immunotherapeutic strategy. In addition, TITE contain damage-associated molecular patterns released from cancer cells, as a function of BAT mediated killing of tumor cells, likely to promote maturation of dendritic cells and cross-priming of cytotoxic T cells [8]. In a 3D matrigel tumorsphere model, TITE induced cytotoxic activity against variety of tumor cells and promoted activation and proliferation of immune cells in the tumor microenvironment (TME). The therapeutic advantages of TITE are manifold. For instance, the administration of TITE eliminates the waiting time associated with the preparation and activation and expansion of a patient's T cells prior to treatment initiation. Further, since TITE can be generated from normal, healthy donor T cells, administration of TITE can be more potent and effective than use of a patient's own T cells. TITE can serve as a ready, off-the-shelf product. Also, TITE can be cost effective compared to cellular therapy. Thus, the presently disclosed subject matter is not only more effective by targeting “tumor” and altering the “TME” but will also overcome the challenges of tumor heterogeneity and immune tolerance by modulating the tumor infiltrating suppressor cells of TME. See also FIGS. 5A and 5B.

Most therapeutic approaches are based on targeting a tumor or a single component of the tumor supporting microenvironment that eventually results in cancer recurrence. The presently disclosed subject matter provides a broad impact and transforms current therapy by simultaneously targeting the tumor and multiple components of the tumor supporting factors while supporting anti-tumor immune responses. The presently disclosed approach of using TITE not only targets cancer cells, “cancer stem like cells,” and immune suppressor cells in the microenvironment but will induce activation and proliferation of immune effector cells that can lead to self-sustained anti-tumor immune responses.

III.B. Representative Treatment Methods and Compositions

In some embodiments, the presently disclosed subject matter provides a method for treating cancer in a subject in need thereof. In some embodiments, the method comprises administering to the subject an effective amount of a composition comprising Tumor-Targeting Effectors (TITE) derived from a culture comprising a bispecific antibody (BiAb) armed activated T cell (BAT) and a cancer cell, to thereby treat cancer in the subject.

In some embodiments, the presently disclosed subject matter provides a pharmaceutical composition comprising, consisting essentially of, or consisting of an effective amount of a composition comprising Tumor-Targeting Effectors (TITE) derived from a culture comprising a BiAb armed activated T cell (BAT) and a cancer cell.

In some embodiments, the presently disclosed subject matter provides a use of a pharmaceutical composition comprising, consisting essentially of, or consisting of an effective amount of a composition comprising Tumor-Targeting Effectors (TITE) derived from a culture comprising a BiAb armed activated T cell (BAT) and a cancer cell to treat cancer in a subject in need thereof.

In some embodiments, the presently disclosed subject matter provides a use of an effective amount of a composition comprising Tumor-Targeting Effectors (TITE) derived from a culture comprising a BiAb armed activated T cell (BAT) and a cancer cell for the preparation of a medicament to treat cancer in a subject in need thereof.

In some embodiments, the culture comprises an about 16 hour culture to an about 48 hour culture comprising a BiAb armed activated T cell (BAT) and a cancer cell. In some embodiments, the culture is selected from the group including but not limited to an about 16, about 20, about 24, about 28, about 32, about 36, about 40, about 44, and about 48 hour culture.

Any suitable or desired cancer cell as would be apparent to one or ordinary skill in the art upon a review of the instant disclosure can be employed. In some embodiments, the cancer cell is from a cancer selected from the group comprising a breast cancer, a pancreatic cancer, a prostate cancer, a brain cancer, a lung cancer, a ovarian cancer, a liver cancer, a leukemia (such as but not limited to acute myelogenous leukemia or acute lymphoblastic leukemia), non-Hodgkin's lymphoma, and multiple myeloma.

Any suitable or desired T cell or T cell effector as would be apparent to one or ordinary skill in the art upon a review of the instant disclosure can be employed. In some embodiments, the T Cell or T cell effector is selected from the group comprising peripheral blood mononuclear cells, unfractionated CD3+ T cells, CD4+ T cells, CD8+ T cells, and combinations thereof. By way of example and not limitation, other T cell or T cell effectors include but are not limited to CD45RO+ T cells, CD45RA+ T cells, and CD3+CD56+ T cells, Tumor infiltrating lymphocytes, CAR-T cells engaging specific leukemia or solid tumors.

In some embodiments, the TITE are prepared by culturing bispecific antibody (BiAb) armed activated T cells with tumor cells at a ratio of T cell effector:tumor cell ranging from about 10:1 to about 50:1, including about 10:1, about 15:1, about 20:1, about 25:1, about 30:1, about 35:1, about 40:1, about 45:1, and about 50:1. In some embodiments, the culture comprises a basal medium. Other representative media and media ingredients are described in the EXAMPLES and/or would be apparent to one of ordinary skill in the art upon a review of the instant disclosure, including but not limited to known and/or commercially available media. By way of example and not limitation, other media and media components include but are not limited to RPMI 1640, Ex vivo 15, Ex Vivo 20, Aim V, CTS OpTmizer T-Cell Expansion SFM, LymphoONE, and/or other T cell culture or equivalent and other complete media in the presence or absence of serum, such as about 2 to about 10% fetal calf serum or human serum, including about 2%, about 3%, about 4%, about 5%, about 6%, about 7%, about 8%, about 9%, and about 10% serum, or artificial serum components (serum free media). In some embodiments, the culture comprises a media comprising RPMI1640 supplemented with 2% human serum. Commercial sources for media include Thermo Fisher Scientific (Hampton, N.H.), MilliporeSigma (Burlington, Mass.) and Sigma-Aldrich (St. Louis, Mo.). Ex vivo 15 and Ex vivo 20 are commercially available under the trademarks X-VIVO™ 15 and X-VIVO™ 20 (Lonza Walkersville, Inc., Walkersville, Md.); Aim V is commercially available under the trademark AIM V™ (Life Technologies Corporation, Carlsbad, Calif.), CTS OpTmizer T-Cell Expansion SFM is commercially available under the trademark CTS™ OpTmizer™ T Cell Expansion SFM (Life Technologies Corporation, Carlsbad, Calif.), and LymphoOne is commercially available under the trademark LYMPHOONE™ (Takara Bio Inc., Kusatsu, Japan).

Any suitable or desired bispecific antibody as would be apparent to one of ordinary skill in the art upon a review of the instant disclosure can be employed. In some embodiments, the bispecific antibody used to arm the activated T cell is selected from the group including but not limited to a chemically heteroconjugated bispecific antibody or recombinant bispecific antibodies of any configuration (univalent, bivalent, or multi-valent bispecific antibodies directed at T cells and at the cancer or tumor antigen).

Any suitable or desired approach for producing activated T cells as would be apparent to one of ordinary skill in the art upon a review of the instant disclosure can be employed. Representative approaches are provided in the EXAMPLES. In some embodiments, the activated T cells are produced from an apheresis product. In some embodiments, the activated T cells are produced from an apheresis product by anti-CD3 stimulation (such as through the use of a soluble OKT3 dose of 20 ng/ml) in the presence of IL-2. In some embodiments, co-stimulated T cells are produced from an apheresis product by co-stimulation with anti-CD³/anti-CD28 coated beads in the presence or absence of IL-2 (5-200 IU/ml, optionally 20-200 IU/ml, including 5, 10, 15, 20, 25, 30, 35, 40, 45, 50, 55, 60, 65, 70, 75, 80, 85, 90, 95, 100, 105, 110, 115, 125, 130, 135, 140, 145, 150, 155, 160, 165, 170, 175, 180, 185, 190, 195, 200 IU/ml), such as at bead to cell ratios from 1:3 to 3:1. Alternatively, co-stimulated T cells or T cell subsets are co-stimulated with anti-CD³/anti-CD²/anti-CD28 coated beads in the presence or absence of IL-2, with IL-2 in the amounts mentioned immediately above when present.

In some embodiments, the TITE have a profile comprising secreted agents selected from the group comprising a Th1 cytokine, a proliferation inducing cytokine, a Th2 cytokine, a chemokine, a growth factor, and any combination thereof. In some embodiments the Th1 cytokine is selected from the group comprising IFN-γ, TNF-α, Granzyme B, GM-CSF, G-CSF, and combinations thereof; the proliferation inducing cytokine is selected from the group comprising Flt3L, IL-2, IL-3, and combinations thereof; the Th2 cytokine is selected from the group comprising IL-10, IL-5, IL-6, IL-13, and combinations thereof; and/or the growth factor is selected from the group comprising CD40L, VEGF, PDGF-AA, and combinations thereof.

In some embodiments, the composition comprising TITE comprises an amount of TITE ranging from about 5% to about 50% by volume or weight, including about 5%, about 10%, about 15%, about 20%, about 25%, about 30%, about 35%, about 40%, about 45%, and about 50% by volume or weight. In some embodiments, the composition comprising TITE comprises a fraction, such as a soluble fraction, comprising components of a molecular weight (e.g., an average molecular weight) of less than about 50 kDa. In some embodiments, the composition comprising TITE comprises a fraction, such as a soluble fraction, comprising components of a molecular weight (e.g., an average molecular weight) ranging from about 10 kiloDaltons (kDa) to about 50 kDa, including about 10 kDa, about 15 kDa, about 20 kDa, about 25 kDa, about 30 kDa, about 35 kDa, about 40 kDa, about 45 kDa, and about 50 kDa.

In some embodiments, the composition comprising TITE comprises a miRNA. In some embodiments, the miRNA is selected from the group including, but not limited to, miR-16-5p, miR-17-5p, miR-195-5p, miR-20a-5p, miR-93-5p, miR-155, miR-181a-5p, miR-181c-5p, miR186-5p, and miR-106a-5p. Representative sequences are disclosed herein below.

The presently disclosed subject matter is also directed to methods of administering the compositions of the presently disclosed subject matter to a subject.

Pharmaceutical compositions comprising the present composition comprising TITE are administered to a subject in need thereof by any number of routes including, but not limited to, topical, oral, intravenous, intramuscular, intra-arterial, intramedullary, intrathecal, intraventricular, transdermal, subcutaneous, intraperitoneal, intranasal, enteral, topical, sublingual, or rectal approaches.

In accordance with one embodiment, a method for treating a subject in need of such treatment is provided. The method comprises administering a pharmaceutical composition comprising at least one composition of the presently disclosed subject matter to a subject in need thereof. Compositions provided by the methods of the presently disclosed subject matter can be administered with known compounds or other medications as well.

By way of representative, non-limiting example, the pharmaceutical compositions useful for practicing the presently disclosed subject matter may be administered to deliver a dose of a composition comprising TITE, which can be concentrated to for example a 10× concentration after isolation as follows: 10× concentrated TITE or equivalent in an amount ranging from about 1 ml/kg/day to about 20 ml/kg/day, from once per week to 3 times per week for 1 month. Retreatment with would recycle the effective, non-toxic dose on the schedule that inhibits the tumor or hematologic malignancy. This representative dosing information references the breast cancer model disclosed herein, with a 30 gram mouse receiving 150 mcl or 0.150 ml per 30 gram mouse translates (multiple 33.33 mice in 1 kg) into 4.95 ml/kg to provide a 3×/week for 3 week dose that inhibited tumor growth. However, based on the instant disclosure and the level of skill in the art, additional dosage amounts and ranges can be established without undue experimentation using ordinary skill in the art.

The presently disclosed subject matter encompasses the preparation and use of pharmaceutical compositions comprising a composition comprising TITE useful for treatment of the diseases and disorders disclosed herein as an active ingredient. Such a pharmaceutical composition can consist of the active ingredient alone, in a form suitable for administration to a subject, or the pharmaceutical composition can comprise the active ingredient and one or more pharmaceutically acceptable carriers, one or more additional ingredients, or some combination of these. The active ingredient can be present in the pharmaceutical composition in the form of a physiologically acceptable ester or salt, such as in combination with a physiologically acceptable cation or anion, as is well known in the art.

As used herein, the term “physiologically acceptable” ester or salt means an ester or salt form of the active ingredient which is compatible with any other ingredients of the pharmaceutical composition, which is not deleterious to the subject to which the composition is to be administered.

The compositions of the presently disclosed subject matter can comprise at least one active ingredient, one or more acceptable carriers, and optionally other active ingredients or therapeutic agents.

Pharmaceutically acceptable carriers include physiologically tolerable or acceptable diluents, excipients, solvents, or adjuvants. The compositions are in some embodiments sterile and nonpyrogenic. Examples of suitable carriers include, but are not limited to, water, normal saline, dextrose, mannitol, lactose or other sugars, lecithin, albumin, sodium glutamate, cysteine hydrochloride, ethanol, polyols (propylene glycol, polyethylene glycol, glycerol, and the like), vegetable oils (such as olive oil), injectable organic esters such as ethyl oleate, ethoxylated isosteraryl alcohols, polyoxyethylene sorbitol and sorbitan esters, microcrystalline cellulose, aluminum methahydroxide, bentonite, kaolin, agar-agar and tragacanth, or mixtures of these substances, and the like.

The pharmaceutical compositions can also contain minor amounts of nontoxic auxiliary pharmaceutical substances or excipients and/or additives, such as wetting agents, emulsifying agents, pH buffering agents, antibacterial and antifungal agents (such as parabens, chlorobutanol, phenol, sorbic acid, and the like). Suitable additives include, but are not limited to, physiologically biocompatible buffers (e.g., tromethamine hydrochloride), additions (e.g., 0.01 to 10 mole percent) of chelants (such as, for example, DTPA or DTPA-bisamide) or calcium chelate complexes (as for example calcium DTPA or CaNaDTPA-bisamide), or, optionally, additions (e.g., 1 to 50 mole percent) of calcium or sodium salts (for example, calcium chloride, calcium ascorbate, calcium gluconate or calcium lactate). If desired, absorption enhancing or delaying agents (such as liposomes, aluminum monostearate, or gelatin) can be used. The compositions can be prepared in conventional forms, either as liquid solutions or suspensions, solid forms suitable for solution or suspension in liquid prior to injection, or as emulsions. Pharmaceutical compositions according to the presently disclosed subject matter can be prepared in a manner fully within the skill of the art.

The compositions of the presently disclosed subject matter or pharmaceutical compositions comprising these compositions can be administered so that the compositions may have a physiological effect. Administration can occur enterally or parenterally; for example, orally, rectally, intracisternally, intravaginally, intraperitoneally, locally (e.g., with powders, ointments or drops), or as a buccal or nasal spray or aerosol. Parenteral administration is an approach. Particular parenteral administration methods include intravascular administration (e.g., intravenous bolus injection, intravenous infusion, intra-arterial bolus injection, intra-arterial infusion and catheter instillation into the vasculature), peri- and intra-target tissue injection, subcutaneous injection or deposition including subcutaneous infusion (such as by osmotic pumps), intramuscular injection, and direct application to the target area, e.g., intratumoral injection, for example by a catheter or other placement device.

Where the administration of the composition is by injection or direct application, the injection or direct application can be in a single dose or in multiple doses. Where the administration of the compound is by infusion, the infusion can be a single sustained dose over a prolonged period of time or multiple infusions.

The formulations of the pharmaceutical compositions described herein can be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

It will be understood by the skilled artisan that such pharmaceutical compositions are generally suitable for administration to animals of all sorts. Subjects to which administration of the pharmaceutical compositions of the presently disclosed subject matter is contemplated include, but are not limited to, humans and other primates, mammals including commercially and/or socially relevant mammals such as cattle, pigs, horses, sheep, cats, and dogs, birds including commercially and/or socially relevant birds such as chickens, ducks, geese, parrots, and turkeys.

A pharmaceutical composition of the presently disclosed subject matter can be prepared, packaged, or sold in bulk, as a single unit dose, or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the presently disclosed subject matter will vary, depending upon the identity, size, and condition of the subject treated and further depending upon the route by which the composition is to be administered. By way of example, the composition can comprise between 0.1% and 100% (w/w) active ingredient.

In addition to the active ingredient, a pharmaceutical composition of the presently disclosed subject matter can further comprise one or more additional pharmaceutically active agents. Particularly provided additional pharmaceutically active agents include chemotherapeutic agents, antibody drug conjugates, bispecific antibodies, proteasome inhibitors, tyrosine kinase inhibitors (TKIs), immunomodulatory imide drugs (IMids), checkpoint inhibitors (anti-PD1 or anti-PDL1 mAbs) and other cancer therapeutics.

Controlled- or sustained-release formulations of a pharmaceutical composition of the presently disclosed subject matter can be made using conventional technology.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the presently disclosed subject matter are known in the art and described, for example in Gennaro (1990) Remington's Pharmaceutical Sciences, 18th ed., Mack Pub. Co., Easton, Pa., United States of America and/or Gennaro (ed.) (2003) Remington: The Science and Practice of Pharmacy, 20th edition Lippincott, Williams & Wilkins, Philadelphia, Pa., United States of America, each of which is incorporated herein by reference.

The compositions may be administered to an animal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type of cancer being diagnosed, the type and severity of the condition or disease being treated, the type and age of the animal, etc.

Other approaches include but are not limited to nanosizing the composition comprising TITE to be delivered as a nanoparticle intravenously, intraperitoneal injection, or implanted beads with time release of TITE. In some embodiments, the composition comprising TITE is adapted for administration for the treatment of a human cancer patient by injecting dose of TITE by intravenous administration, intrathecal injection, peritoneal injection, or direct injection into the tumor or surround tumor site. In some embodiments, the composition comprising TITE is adapted for administration for the treatment of an animal patient (dogs, cats, cows, horses, and pigs by injecting dose of TITE by intravenous administration, peritoneal injection, or direct injection into the tumor or surround tumor site.

Suitable preparations include injectables, either as liquid solutions or suspensions, however, solid forms suitable for solution in, suspension in, liquid prior to injection, may also be prepared. The preparation may also be emulsified, or the compositions encapsulated in liposomes. The active ingredients are often mixed with excipients which are pharmaceutically acceptable and compatible with the active ingredient. Suitable excipients are, for example, water saline, dextrose, glycerol, ethanol, or the like and combinations thereof. In addition, if desired, the preparation may also include minor amounts of auxiliary substances such as wetting or emulsifying agents, pH buffering agents, and/or adjuvants.

The presently disclosed subject matter also includes a kit comprising the composition of the presently disclosed subject matter and an instructional material which describes administering the composition to a cell or a tissue of a subject. In some embodiments, this kit comprises a (in some embodiments sterile) solvent suitable for dissolving or suspending the composition of the presently disclosed subject matter prior to administering the compound to the subject and/or a device suitable for administering the composition such as a syringe, injector, or the like or other device as would be apparent to one of ordinary skill in the art upon a review of the instant disclosure.

As used herein, an “instructional material” includes a publication, a recording, a diagram, or any other medium of expression which can be used to communicate the usefulness of the composition of the presently disclosed subject matter in the kit for effecting alleviation of the various diseases or disorders recited herein. Optionally, or alternately, the instructional material may describe one or more methods of using the compositions for diagnostic or identification purposes or of alleviation the diseases or disorders in a cell or a tissue of a mammal. The instructional material of the kit of the presently disclosed subject matter can, for example, be affixed to a container which contains a composition of the presently disclosed subject matter or be shipped together with a container which contains the composition. Alternatively, the instructional material can be shipped separately from the container with the intention that the instructional material and the composition be used cooperatively by the recipient.

In accordance with the presently disclosed subject matter, as described above or as discussed in the EXAMPLES below, there can be employed conventional chemical, cellular, histochemical, biochemical, molecular biology, microbiology, recombinant DNA, and clinical techniques which are known to those of skill in the art. Such techniques are explained fully in the literature. See for example, Sambrook et al. (1989) Molecular Cloning: A Laboratory Manual, Cold Spring Harbor Laboratory Publications, Cold Spring Harbor, N.Y., United States of America; Glover (1985) DNA Cloning: A Practical Approach. Oxford Press, Oxford; Gait (1984) Oligonucleotide Synthesis: A Practical Approach, IRL Press, Oxford, England; Harlow & Lane, 1988, Antibodies, A Laboratory Manual, Cold Spring Harbor Publications, New York; Roe et al. (1996) DNA Isolation and Sequencing: Essential Techniques, John Wiley, New York, N.Y., United States of America; and Ausubel et al. (1995) Current Protocols in Molecular Biology, Greene Publishing.

### III.C. Antibody Formats and Preparation Thereof

Any suitable bispecific antibody and technique for the production thereof as would be apparent to one of ordinary skill in the art upon a review of the instant disclosure falls within the scope of the presently disclosed subject matter. In some embodiments, the presently disclosed subject matter employs bispecific antibodies (BiAbs) produced by chemical joining of two monoclonal antibodies. Examples of bispecific antibodies and techniques for producing bispecific antibodies are known the art and have been described in several reviews, along with their respective cancer antigens and T cell antigens. Representative reviews include Thakur, A., and Lum, L. G.: Cancer therapy with bispecific antibodies: Clinical experience. Current Opinion and Molecular Therapeutics 12:340-349, 2010; Lum, L. G., and Thakur, A.: Bispecific Antibodies for Arming Activated T Cells and Other Effector Cells for Tumor Therapy. Book Chapter in: Bispecific Antibodies. Kontermann, R. E. (ed). Germany: Springer Heidelberg, 2011, pp. 243-271; Lum, L. G., and Thakur, A.: Targeting T Cells with Bispecific Antibodies for Cancer Therapy: A Review. BioDrugs 25:365-379, 2011; and Thakur, A., Huang, M., Lum, L. G.: Bispecific antibody based therapeutics: Strengths and challenges. Blood Reviews, 2018 (Impact 6.6). Representative BiAbs include but are not limited to whole IgG-based BiAbs, trifunctional BiAbs, BiAb Format based on single-chain variable fragment. Representative U.S. patents relating to BiAbs and production thereof include U.S. Pat. Nos. 10,550,193; 10,519,247; 10,294,300; 10,239,951; and 10,179,819, each of which is herein incorporated by reference in its entirety.

In some embodiments, one or more antibodies or fragments thereof are used. In some embodiments, one or both antibodies are single chain, monoclonal, bi-specific, synthetic, polyclonal, chimeric, human, or humanized, or active fragments or homologs thereof. In some embodiments, the antibody binding fragment is scFV, F(ab′)₂, F(ab)₂, Fab′, or Fab. Fragments within the scope of the term “antibody” include those produced by digestion with various proteases, those produced by chemical cleavage and/or chemical dissociation and those produced recombinantly, so long as the fragment remains capable of specific binding to a target molecule. Among such fragments are Fab, Fab′, Fv, F(ab′)₂, and single chain Fv (scFv) fragments. In some embodiments, the specific binding molecule is a single-chain variable (scFv). The specific binding molecule or scFv may be linked to other specific binding molecules (for example other scFvs, Fab antibody fragments, chimeric IgG antibodies (e.g., with human frameworks)) or linked to other scFvs of the presently disclosed subject matter so as to form a multimer which is a multi-specific binding protein, for example a dimer, a trimer, or a tetramer. Bi-specific scFvs are sometimes referred to as diabodies. Fragments within the scope of the term “antibody” include those produced by digestion with various proteases, those produced by chemical cleavage and/or chemical dissociation and those produced recombinantly, so long as the fragment remains capable of specific binding to a target molecule (i.e., comprise at least one paratope).

Other representative patent documents disclosing techniques relating to antibody production include the following, all of which are herein incorporated by reference in their entireties: PCT International Patent Application Publication Nos. WO 1992/02190 and WO 1993/16185; U.S. Patent Application Publication Nos. 2004/0253645, 2003/0153043, 2006/0073137, 2002/0034765, and 2003/0022244; and U.S. Pat. Nos. 4,816,567; 4,946,778; 4,975,369; 5,001,065; 5,075,431; 5,081,235; 5,169,939; 5,202,238; 5,204,244; 5,225,539; 5,231,026; 5,292,867; 5,354,847; 5,436,157; 5,472,693; 5,482,856; 5,491,088; 5,500,362; 5,502,167; 5,530,101; 5,571,894; 5,585,089; 5,587,458; 5,641,870; 5,643,759; 5,693,761; 5,693,762; 5,712,120; 5,714,350; 5,766,886; 5,770,196; 5,777,085; 5,821,123; 5,821,337; 5,869,619; 5,877,293; 5,886,152; 5,895,205; 5,929,212; 6,054,297; 6,180,370; 6,407,213; 6,548,640; 6,632,927; 6,639,055; 6,750,325; and 6,797,492.

III.D. Representative Preparation Methods

In some embodiments, the presently disclosed subject matter provides a method of preparing a composition for treating cancer. In some embodiments, the method comprises (a) culturing T cells and cancer cells with a bispecific antibody to provide a culture comprising a complex comprising cancer cells and activated T cells (ATC) wherein one binding domain of the bispecific antibody binds to an antigen on the T cells and a second binding domain of the bispecific antibody binds an antigen on the cancer cells; and (b) isolating media from the culture, wherein the media comprises BAT Induced Tumor-Targeting Effectors (TITE), to thereby provide a composition for treating cancer. In some embodiments, the isolated media is a conditioned media. In some embodiments, the method further comprises concentrating, isolating, and/or purifying the composition comprising TITE. Any suitable approach for concentration, isolating and/or purifying the composition comprising TITE can be employed. Representative approaches are disclosed in the EXAMPLES below. Other approaches include but are not limited to nanosizing the composition comprising TITE to be delivered as a nanoparticle intravenously, intraperitoneal injection, or implanted beads with time release of TITE. In some embodiments, the composition comprising TITE is adapted for administration for the treatment of a human cancer patient by injecting dose of TITE by intravenous administration, intrathecal injection, peritoneal injection, or direct injection into the tumor or surround tumor site. In some embodiments, the composition comprising TITE is adapted for administration for the treatment of an animal patient (dogs, cats, cows, horses, and pigs by injecting dose of TITE by intravenous administration, peritoneal injection, or direct injection into the tumor or surround tumor site.

In some embodiments, the culture comprises an about 16 hour culture to an about 48 hour culture comprising a bispecific antibody armed activated T cell (BAT) and a cancer cell. In some embodiments, the culture is selected from the group including but not limited to about an about 16, 20, 24, 28, 32, 36, 40, 44, and an about 48 hour culture.

Any suitable or desired cancer cell as would be apparent to one or ordinary skill in the art upon a review of the instant disclosure can be employed. In some embodiments, the cancer cell is from a cancer selected from the group comprising a breast cancer, a pancreatic cancer, a prostate cancer, a brain cancer, a lung cancer, a ovarian cancer, a liver cancer, a leukemia (such as but not limited to acute myelogenous leukemia or acute lymphoblastic leukemia), non-Hodgkin's lymphoma, and multiple myeloma.

Any suitable or desired T cell or T cell effector as would be apparent to one or ordinary skill in the art upon a review of the instant disclosure can be employed or present in the culture media. In some embodiments, the T Cell or T cell effector is selected from the group comprising peripheral blood mononuclear cells, CD4+ T cells, CD8+ T cells, and combinations thereof. By way of example and not limitation, other T cell or T cell effectors include but are not limited to unfractionated CD3+ T cells. CD4+ T cells, CD8+ T cells, and combinations thereof. By way of example and not limitation, other T cell or T cell effectors include but are not limited to CD45RA+ T cells, CD45RO+ T cells, Tumor infiltrating lymphocytes, CAR-T cells engaging specific leukemia or solid tumors.

In some embodiments, the TITE are prepared by culturing bispecific antibody (BiAb) armed T cells and tumor cells at a ratio T cell effector:tumor cell ranging from about 10:1 to about 50:1, including about 10:1, 15:1, 20:1, 25:1, 30:1, 35:1, 40:1, 45:1, and about 50:1. In some embodiments, the culture comprises a basal medium. In some embodiments the medium comprises RPMI1640 supplemented with 2% human serum. Other representative media and media components are described in the EXAMPLES and/or would be apparent to one of ordinary skill in the art upon a review of the instant disclosure. By way of example and not limitation, other media and media ingredients include but are not limited to including but not limited to known and/or commercially available media. By way of example and not limitation, other media and media components include but are not limited to RPMI 1640, Ex vivo 15, Ex Vivo 20, Aim V, CTS OpTmizer T-Cell Expansion SFM, LymphoONE, and/or other T cell culture or equivalent and other complete media in the presence or absence of serum, such as about 2 to about 10% fetal calf serum or human serum, including about 2%, about 3%, about 4%, about 5%, about 6%, about 7%, about 8%, about 9%, and about 10% serum by weight or by volume, or artificial serum components (serum free media). In some embodiments, the culture comprises a media comprising RPMI1640 supplemented with 2% human serum.

Any suitable or desired bispecific antibody as would be apparent to one of ordinary skill in the art upon a review of the instant disclosure can be employed. In some embodiments, the bispecific antibody used to arm the activated T cell is selected from the to group including but not limited to a chemically heteroconjugated bispecific antibody or recombinant bispecific antibodies of any configuration (univalent, bivalent, or multi-valent bispecific antibodies directed at T cells and at the cancer or tumor antigen),

Any suitable or desired approach for producing activated T cells as would be apparent to one of ordinary skill in the art upon a review of the instant disclosure can be employed. Representative approaches are provided in the EXAMPLES. In some embodiments, the activated T cells are produced from an apheresis product. In some embodiments, co-stimulated T cells are produced from an apheresis product by co-stimulation with anti-CD³/anti-CD28 coated beads in the presence or absence of IL-2 (5-200 IU/ml, optionally 20-200 IU/ml, including 5, 10, 15, 20, 25, 30, 35, 40, 45, 50, 55, 60, 65, 70, 75, 80, 85, 90, 95, 100, 105, 110, 115, 125, 130, 135, 140, 145, 150, 155, 160, 165, 170, 175, 180, 185, 190, 195, 200 IU/ml), such as at bead to cell ratios from 1:3 to 3:1. Alternatively, co-stimulated T cells or T cell subsets are co-stimulated with anti-CD³/anti-CD²/anti-CD28 coated beads in the presence or absence of IL-2, with IL-2 in the amounts mentioned immediately above when present.

In some embodiments, the TITE have a profile comprising secreted agents selected from the group comprising a Th1 cytokine, a proliferation inducing cytokine, a Th2 cytokine, a chemokine, a growth factor, and any combination thereof. In some embodiments the Th1 cytokine is selected from the group comprising IFN-γ, TNF-α, Granzyme B, GM-CSF, G-CSF, and combinations thereof; the proliferation inducing cytokine is selected from the group comprising Flt3L, IL-2, IL-3, and combinations thereof; the Th2 cytokine is selected from the group comprising IL-10, IL-5, IL-6, IL-13, and combinations thereof; and/or the growth factor is selected from the group comprising CD40L, VEGF, PDGF-AA, and combinations thereof.

In some embodiments, the composition comprising TITE comprises an amount of TITE ranging from about 5% to about 50% by volume or weight, including about 5%, about 10%, about 15%, about 20%, about 25%, about 30%, about 35%, about 40%, about 45%, and about 50% by volume or weight. In some embodiments, the composition comprising TITE comprises a soluble fraction comprising components of a molecular weight of less than about 50 kDa. In some embodiments, the composition comprising TITE comprises a soluble fraction comprising components of a molecular weight ranging from about 10 kiloDaltons (kDa) to about 50 kDa, including about 10 kDa, about 15 kDa, about 20 kDa, about 25 kDa, about 30 kDa, about 35 kDa, about 40 kDa, about 45 kDa, and about 50 kDa.

In some embodiments, the composition comprising TITE comprises a miRNA. In some embodiments, the miRNA is selected from the group including, but not limited to, miR-16-5p, miR-17-5p, miR-195-5p, miR-20a-5p, miR-93-5p, miR-155, miR-181a-5p, miR-181c-5p, miR186-5p, and miR-106a-5p.

Sequences for miRNA

| miRID | Species | Sequence |
|---|---|---|
| miR-16-5p | Human | UAGCAGCACGUAAAUAUUGGCG |
| | and Mouse | (SEQ ID NO: 1) |
| miR-17-5p | Human | CAAAGUGCUUACAGUGCAGGUAG |
| | and Mouse | (SEQ ID NO: 2) |
| miR-195-5p | Human | UAGCAGCACAGAAAUAUUGGC |
| | and Mouse | (SEQ ID NO: 3) |
| miR-20a-5p | Human | UAAAGUGCUUAUAGUGCAGGUAG |
| | and Mouse | (SEQ ID NO: 4) |
| miR-93-5p | Human | CAAAGUGCUGUUCGUGCAGGUAG |
| | and Mouse | (SEQ ID NO: 5) |
| miR-155 | Human | UUAAUGCUAAUCGUGAUAGGGGUU |
| | | (SEQ ID NO: 6) |
| | Mouse | UUAAUGCUAAUUGUGAUAGGGGU |
| | | (SEQ ID NO: 7) |
| miR-181a-5p | Human | AACAUUCAACGCUGUCGGUGAGU |
| | and Mouse | (SEQ ID NO: 8) |
| miR-181c-5p | Human | AACAUUCAACCUGUCGGUGAGU |
| | and Mouse | (SEQ ID NO: 9) |
| miR-186-5p | Human | CAAAGAAUUCUCCUUUUGGGCU |
| | and Mouse | (SEQ ID NO: 10) |
| miR-106a-5p | Human | AAAAGUGCUUACAGUGCAGGUAG |
| | and Mouse | (SEQ ID NO: 11) |

### IV. EXAMPLES

The presently disclosed subject matter will be now be described more fully hereinafter with reference to the accompanying EXAMPLES, in which representative embodiments of the presently disclosed subject matter are shown. The presently disclosed subject matter can, however, be embodied in different forms and should not be construed as limited to the embodiments set forth herein. Rather, these embodiments are provided so that this disclosure will be thorough and complete, and will fully convey the scope of the presently disclosed subject matter to those skilled in the art.

### Materials and Methods for Examples

Cell Lines. The human breast cancer (MDA-MB-231, BT-20, SK-BR-3, MCF-7) and pancreatic cancer cell lines (MiaPaCa-2, COLO-356) were maintained in DMEM culture media (Lonza Inc., Allendale, N.J.) supplemented with 10% FBS (Lonza Inc.), 2 mM L-glutamine (Invitrogen, Carlsbad, Calif.), 50 units/ml penicillin, and 50 μg/ml streptomycin (Invitrogen).

Expansion and Generation of activated T cells (ATC). T cells from PBMC were activated with 20 ng/ml of OKT3 and expanded in 100 IU/ml of IL-2 for 14 days in RPMI-1640 supplemented with 10% FBS as previously described [3]. Harvested ATC were armed with bispecific antibody (anti-CD3×anti-HER2 [HER2Bi] or anti-CD3×anti-EGFR [EGFRBi]) at pre-optimized concentration of 50 ng/10⁶ ATC as previously described [5]. See also, U.S. Patent Application Publication No. 2006/0034767 (anti-CD3×anti-HER2 [HER2Bi] example) and U.S. Patent Application Publication No. 2018/0243341 (anti-CD3×anti-EGFR [EGFRBi] example); herein incorporated by reference in their entireties. Generation of Conditioned Media. The TITE was prepared by overnight culture of 1×10⁶ tumor cells (T) and 25×10⁶ of HER2 BATs or EGFR BATs (25:1 E/T ratio) in 5 ml RPMI-1640 supplemented with 2% human serum overnight followed by collecting and centrifuging the culture supernatant to remove cells and cellular debris. Control CMs were prepared using 1×10⁶ tumor cells (T-CM) in 5 ml DMEM media supplemented with 2% human serum or 25×10⁶ HER2 BATs or EGFR BATs (B-CM) in 5 ml RPMI-1640 media supplemented with 2% human serum. The TITE was prepared and tested from at least 10-12 normal donor ATC either alone or in combination (i.e., some experiments pooled TITE from 3-5 donors). TITE was either used fresh or frozen at −70° C. for later use. For animal studies TITE was concentrated 10× using 3 kDa cutoff Millipore centrifugal devices.

3D Culture in Matrigel. Typically, tumor cells were adjusted to a concentration of 5000 cells/ml in DMEM culture media and overlaid on a solidified layer of matrigel as described previously (9, 10). Briefly, wells were coated with 100% matrigel in 0.25-ml aliquots in 24-well plates and allowed to solidify by incubating at 37° C. for 30 minutes. Cancer cells were then seeded onto the matrigel base as a single-cell suspension in the medium containing 2% matrigel. Once tumor spheres were formed (5-7 days), PBMC or MDSC were added at 10:1 ratio (10 PBMC to 1 tumor cell) and cultured for additional 5-7 days in the presence or absence of TITE, B-CM and T-CM.

Cytotoxicity Assay. Cytotoxic activity of TITE against tumor spheres on matrigel was measured by MTT and cytotoxicity of TITE against multiple adherent tumor cells in 2D culture was assessed by 51Cr release assay as previously described [7]. For MTT, at the end of the incubation, 3-(4,5-dimethylthiazolyl-2)-2,5-diphenyltetrazolium bromide (MTT) was added (40 μL/well of 5 mg/mL MTT in PBS in 96-well plate) to each well and incubated in the dark for 3 h at 37° C. After removal of the medium, the dye crystals formed in viable cells were dissolved in isopropanol (or SDS for 3D cultures) and detected by reading the absorption at 595 nm in the Tecan Ultra plate reader. Experiments were repeated three times in quadruplicate wells to ensure reproducibility.

Real Time Monitoring of Cytotoxicity by xCELLigence system. In Real Time Cell Analysis (RTCA) system, proliferation or cytotoxicity is measured by cellular impedance readout as Cell Index (CI) to monitor real-time changes in cell number. This is derived from the relative impedance changes corresponding to cellular coverage of the electrode sensors, normalized to baseline impedance values with medium only. Cell attachment was monitored using the RTCA software until the plateau phase was reached, which was usually after approximately 22-24 h before adding effector cells or TITE. We used breast (MB231, MCF-7) and pancreatic (MiaPaCa-2) cancer cell lines for xCELLigence RTCA as targets and TITE or BATs as effectors. The target cells (10-20,000 cells/well as optimized for each cell line) were plated in 96 well E-Plates followed by adding either 5% TITE alone or 5% TITE to prime tumor cells for 24 h before adding BATs at 2:1 E:T ratio. The target cell impedance signal was monitored for 120 h. Untreated targets or effectors without targets served as controls. To analyze the acquired data, CI values were exported and percentage of lysis was calculated in relation to the control cells lacking any effector cells.

Effect of TITE on Immune Cells. We accessed the effect of TITE on the phenotype of immune cells, immune cell activation (PBMC) and immune suppressor cells (MDSC, Tregs) when co-cultured in 3D matrigel with tumor cells by flow cytometry for 3 days. For phenotypic changes, non-adherent cells were collected, matrigel was digested using dispase to collect tumor cells, tumor associated MDSC or other cells, and washed with FACS buffer (0.2% BSA in PBS). Cells collected prior to digestion were pooled with matrigel digested single cell suspension before staining. Cells were stained for 30 minutes on ice with mixtures of fluorescently conjugated mAbs or isotype-matched controls, washed twice with FACS buffer and analyzed. Antibodies used for staining include: anti-CD45, -CD3, -CD4, -CD8, -CD69, -41BB, -ICOS, -OX40, -PD1, -CD152, -CD11b, -CD14, -CD15, -CD33, -HLA-DR, -CD133, -CD44, -CD24, -EpCAM, -CD56, -CD19, -CD20, -CD80, -CD86 (BD Biosciences San Jose, Calif.).

Cells were analyzed on a FACScalibur (BD Biosciences) and data were analyzed using FlowJo software (BD Biosciences). T cell activation was analyzed by CD69+CD4 and CD8 T cells; costimulatory receptor expression by gating on 41BB/ICOS/OX40 on CD45+/CD3+/CD4+ or CD45+/CD3+/CD8+ T cells and co-inhibitory receptor expression by gating on CD152/PD-1 on CD45+/CD3+/CD4+ or CD45+/CD3+/CD8+ T cells. For MDSC, cells were gated on CD11b+/CD33+ population and analyzed for CD14+/HLA-DR- and CD15+/HLA−DR− expression. For mature APC, cells were gated on CD14− population and analyzed for CD80+/CD86+ cells. Cancer stem like cells (CSC) were gated on EpCAM+/CD133+ population and analyzed for CD44hi/CD24lo/− among MB-231 cells. Isolation of cancer stem like cells. CD133+ cells were isolated using magnetic microbeads (Miltenyi Biotec). CD133+ cells were then cultured in low adherence culture plates at 1000 cells/ml concentration in serum free tumor sphere forming media (Life Technology). Cells that form tumor spheres are likely to be cancer stem like cells (CSC), more that 5% cells showed CSC phenotype using flow cytometry by gating CD133+/EpCAM+ cells to analyze CD44hi and CD24lo/− cells in breast cancer cell lines.

MDSC Generation. We used anti-CD33 magnetic microbeads (Miltenyi Biotec,) to isolate CD33+ cells from co-cultures as previously described [9]. The CD33+ cells were cultured in the presence of 10 ng/ml GM-CSF and 10 ng/ml IL-6 for 7 days, media and cytokines were replace every 2-3 days and purity of cytokine stimulated MDSC was checked by flow cytometry for granulocytic and monocytic MDSC populations. The purity of isolated cell populations was found to be ˜90% by flow cytometry.

Analysis of Conditioned Media Size Based Separation of Conditioned Media for Functional Analysis. TITE was fractionated using different molecular weight cut-off membranes. Small <10 kDa, medium <50 kDa, large >50 kDa molecular weight proteins were separated by Millipore filtration devise. Less than 3 kDa membrane was used to remove all proteins, RNase and DNase digestion was used as RNA/DNA free TITE, heat treated TITE was used as protein, RNA and DNA free TITE. Fractionated TITE was used to narrow down the effector protein pool for functional protein analysis.

Cytokine Profiling of Conditioned Media. Cytokines were quantitated in culture supernatants collected from multiple donors ATC and multiple tumor cell lines in various culture conditions-tumor cells (MB-231, MCF-7, MiaPaCa-2) alone (T-CM), BATs alone (B-CM), tumor+BAT co-cultures (TITE) using a 25-plex human cytokine Bio-Plex Array (Invitrogen, Carlsbad, Calif.) on a Luminex system (Bio-Rad Lab., Hercules, Calif.) as previously described [7, 9]. The limit of detection for these assays is <10 pg/mL based on detectable signal of >2 fold above background (Bio-Rad). Cytokine concentrations were automatically calculated by the BioPlex Manager Software (Bio-Rad).

Phosphorylation-specific Protein Microarray of TITE. Phosphorylation-specific antibody microarrays (Fullmoon Biosystems) were used to see the pattern of up- and down-regulated proteins in T-CM, B-CM and TITE collected after 18 hour of culture. The protein array included 120 phospho-specific antibodies to proteins including in JAK/STAT signaling pathway. The array layout included antibodies against phosphorylated- and unphosphorylated-proteins, each replicated six times, actin and GAPDH served as controls. In brief, proteins were labeled with biotin and placed on pre blocked microarray slides. After washing, detection of total and phosphorylated proteins was performed using Cy3-conjugated streptavidin. Expression of phosphorylated proteins was normalized to corresponding total protein expression from the intensity values obtained. Fold change represent the ratio of phosphorylation in T-CM, B-CM or TITE. Where indicated, protein phosphorylation data were confirmed by Western blot.

Exosomal microRNA Isolation from TITE, RT-PCR, Real Time-PCR and miScript miRNA PCR Array. Micro RNAs (miRNA) from TITE were isolated using Novak culture supernatant miRNA extraction kit (Novak, Canada). Reverse transcription (RT) was performed using the RT primers for each individual miRNA (Life Technologies) according to the manufacturer's instructions. Real-time PCR was performed using a miRVana qRT-PCR kit and PCR primers (Life Technologies) according to the manufacturer's instructions. Human Inflammatory Response miRNA PCR Array: MIHS-105Z mi Script miRNA PCR Array was done by Quigen.

Subcutaneous Breast Cancer Xenograft Model. Eight to ten weeks old female ICR/Scid mice were injected subcutaneously (subQ) with MDA-MB-231 (5×106 cells/mouse) into the left flank. Tumor volume was measured twice a week with a caliper and calculated using the formula: A×B2/2, where A=length of tumor and B=width of tumor. Mice received 20×106 HER2Bi armed ATC (HER2 BATs), or received 150 μl 10×TITE either IV or IT and control group received PBS IV or IT three time a week for 2-3 weeks. At the end of two or three weeks, mice were sacrificed, and tumors were harvested for histopathological analyses and immunohistochemistry for macrophage and granulocyte infiltration.

Collection of Tissue Samples from Mice. The tumor, spleen, liver, heart and lungs were collected and washed in PBS. Tumors were cut into two, one part of the tumor was minced into small pieces and incubated in enzyme cocktail (Miltenyi Biotec) followed by cell dissociation using Miltenyi tissue dissociator. Dissociated cells were passed through a cell strainer and washed three times in RPMI-1640 supplemented with 10% FCS, gentamicin and L-glutamine (complete media). Spleens were homogenized through mincing and passing through a cell strainer to achieve single cell suspensions. Red blood cells were lysed using ACK Lysis Buffer (Cambrex/BioWhittaker). Liver, heart and lungs were analyzed for toxicity. Second part was fixed in buffered formalin followed by paraffin embedding, sectioning and staining.

Statistical Analysis. Quantitative data are presented as the mean of at least three or more independent experiments±standard deviation. A one-way ANOVA was used to determine whether there were statistically significant differences among different conditions within each experiment. Differences between groups were tested via an unpaired, two-tailed t test. For each test, P<0.05 were considered statistically significant.

### Example 1

### TITE Inhibits Tumor Sphere in 3D Cultures

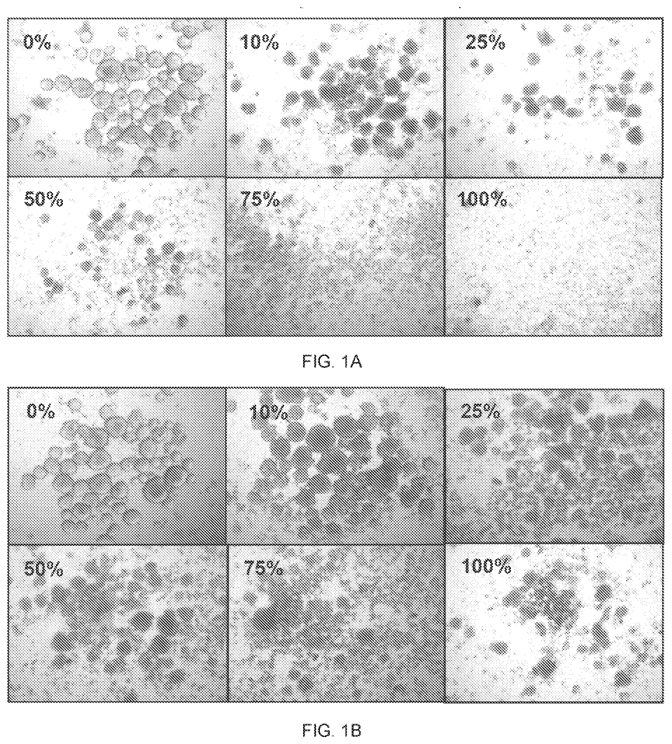

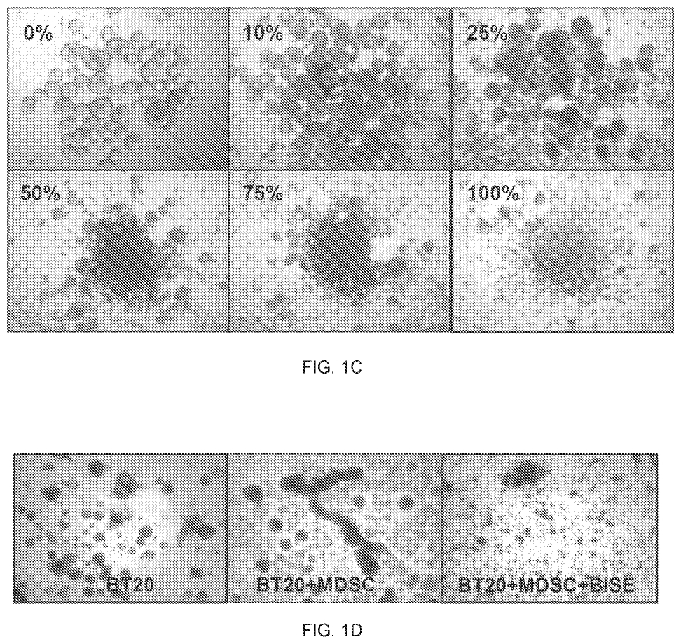

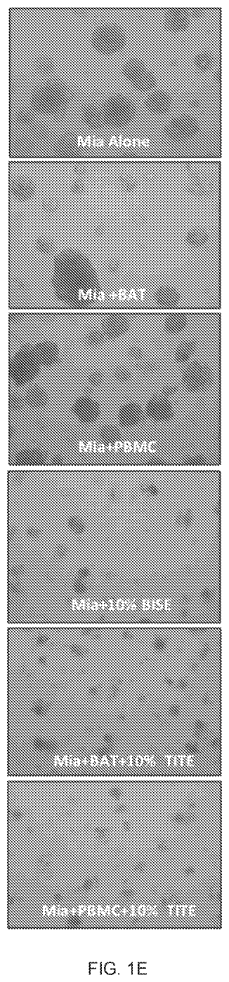

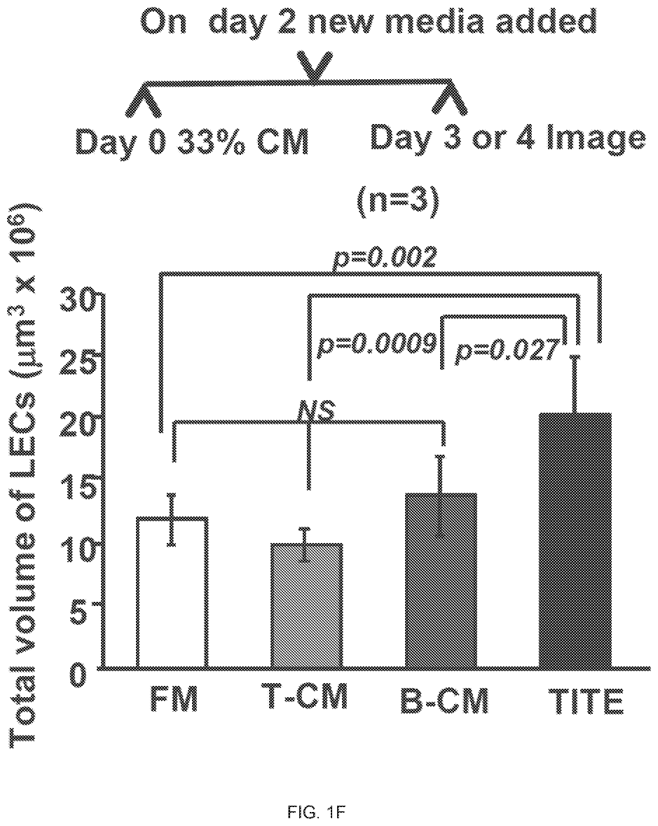

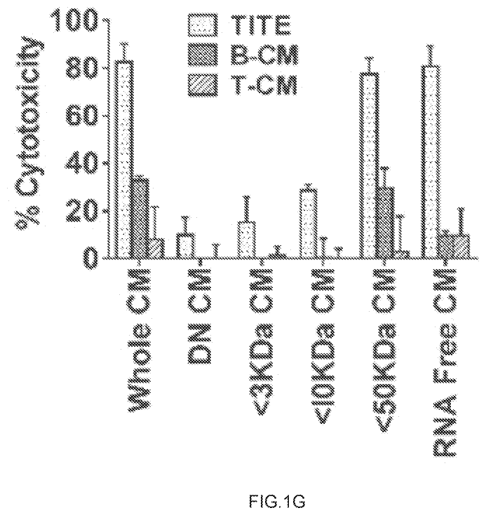

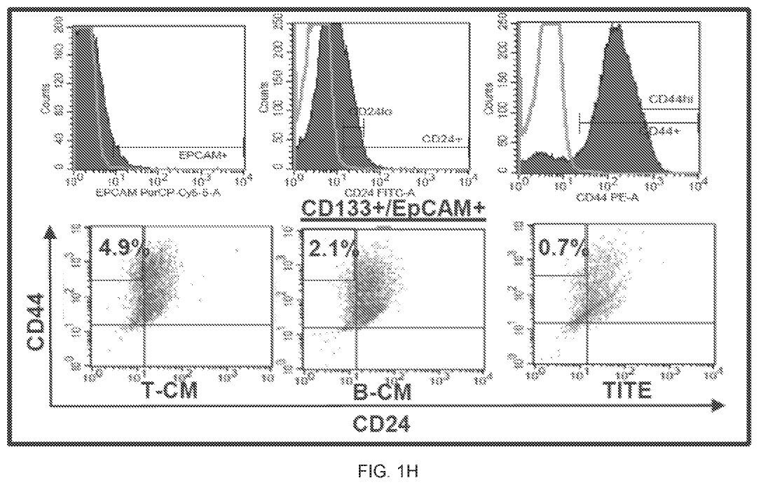

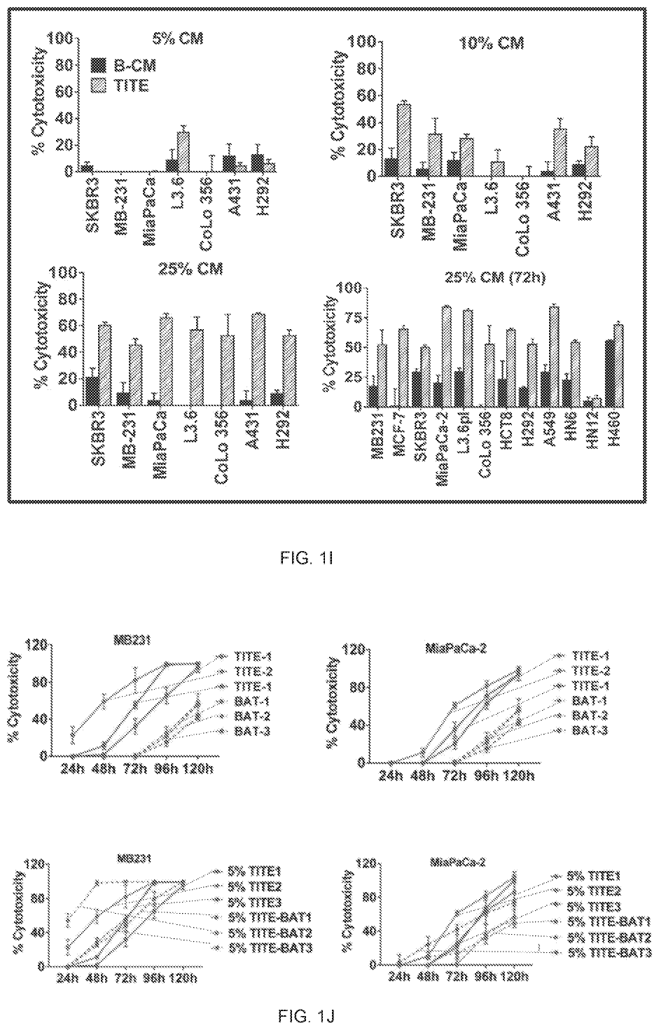

Since Th1 cytokines are released during BATs mediated killing of tumor cells, in this study, Th1 cytokine enriched secretome from tumor+ BATs co-culture (released cytokines/chemokines/growth factors and other mediators in TITE) was investigated for its ability to inhibit the growth of tumor spheres. TITE were prepared from T cell subpopulations, 1) CD3+ T cell, 2) CD4+ T cells and, 3) CD8+ T cells armed with HER2Bi, and co-cultured with tumor cells (MB231) for 24 hours. The breast cancer cell lines-BT20 and MB231 and pancreatic cancer cell line-MiaPaCa-2 were cultured in the presence or absence of various percentage of TITE (0-100%) for 5 days followed by imaging and MTT assay to determine the % viable cells in 3D culture. FIGS. 1A-1C show the microscopic images of tumor spheres incubated with TITE prepared from T cell subsets (FIG. 1A. CD3+ T cell, FIG. 1B. CD4+ T cells and, 3. CD8+ T cells) for 72 hours at indicated TITE concentrations. The TITE prepared from unfractionated activated T cells (CD3+ T cells) showed marked killing (FIG. 1A) of BT20-tumor spheres compared to the TITE generated from CD4+ or CD8+ T cell fractions (FIGS. 1B and 1C). FIG. 1D shows TITE mediated disruption of tumor spheres in the presence of myeloid derived suppressor cells. Similar observations were made for MiaPaCa-2 tumor spheres coculture with BATs or PBMC in the presence or absence of TITE (FIG. 1E).

### Example 2

### Effect of TITE on Breast Cancer Cells by Live Confocal Imaging of 3D Cultures

For confocal imaging, MB231 breast cancer cell line labeled with green fluorescent dye was cultured in Cultrex™ BME on glass cover slips in the presence or absence of TITE (33%) for 3-4 days followed by confocal imaging to see the effect of TITE on breast cancer cells. The TITE showed a dramatic decrease in proliferation of MB231 cells using TITE from either single normal donor BATs or pooled TITE prepared from 3 normal donor BATs compared to control T-CM, B-CM and normal growth media. A remarkable inhibition of 3D cultures of MB231 cells in the presence of TITE was observed, as compared to untreated control, T-CM or B-CM treated cells. Enhanced proliferation of BATs co-cultured with breast cancer cells-MB231 in the presence of TITE was observed and it was confirmed that T cells incubated with TITE show significantly increased proliferation compared to control CMs.

### Example 3

### Effect of TITE on Lymphatic Endothelial Cells (LEC)

Similar to MB231 cells, LEC-tert (kind gift from Dr. Groger) were labeled with green fluorescent dye and cultured in the presence or absence of TITE (33%) for confocal imaging. Intriguingly, the TITE showed a completely reverse effect on LECs by enhancing the proliferation with larger nodes compared to control T-CM, B-CM and normal growth media. Significantly increased LEC proliferation with TITE was observed, as compared to T-CM (p<0.0009), B-CM (p<0.027) and normal growth media (p<0.002). See also FIG. 1F.

### Example 4

### Effect of TITE on MB231 and LEC Co-Cultures

Further, TITE were examined to see if they show a similar effect in a co-culture of MB231 cells and LECs as seen with individual cell type. Indeed, TITE showed reduced proliferation of MB231 (red) and increased proliferation of LEC (green) as observed with each grown separately with TITE. In top and side views of lymphatic endothelial cells (LEC), TITE shows enhanced nodal proliferation of LECs. Thus, a co-culture of MB231 and LECs showed reduced proliferation of MB231 (shown as red in the images) and increased proliferation of LEC (shown as green in the images) in a co-culture of MB231 and LEC, which is consistent with the observation when each grown separately with TITE, suggesting differential cell specific effects of TITE.

### Example 5

### TITE Induce Proliferation of BATs in Co-Culture with MB231 Cells

BATs and MB231 cells cocultured for with MB231 cells 72 h at 10:1 ratio, majority of tumor cells were killed in the culture. However, co-culture of BATs with MB231 cells in the presence of 5% TITE showed increased proliferation of BATs compared to control CMs and fewer tumor spheres in z-stacked images.

### Example 6

### Activity of TITE is Retained in >10 kDa and <50 kDa Molecular Weight Fractions

The present results suggest that the soluble factor(s) between 10 to 50 kDa molecular weight retains cytotoxic activity as shown in FIG. 1G against MB231 cells. Fractions below 3 kDa, <10 kDa or heat treated showed low to no cytotoxic activity. Since functional activity was heat-sensitive, the factor(s) appeared to be protein(s). Likewise, soluble factor(s) between 10 to 50 kDa molecular weight retained immune activating activity.

### Example 7

### TITE Inhibits CSC in 3D Suspension Cultures

CD133+ sorted cancer stem like cells (CSC) from breast cancer cell line MB231, expanded in a ultra-low adherence plates using serum-free DMEM/F12 supplemented with 100 IU/ml penicillin, 100 μg/ml streptomycin, 20 ng/ml human recombinant epidermal growth factor (hrEGF), 10 ng/ml human recombinant basic fibroblast growth factor (hrbFGF), 2% B27 supplement without vitamin A and 1% N2 supplement (Invitrogen, Carlsbad, Calif., USA), after 5 days tumor clusters were cultured in the presence or absence of 25% TITE for 7 days followed by staining for EpCAM, CD44 and CD24. Cells were gated on EpCAM+/CD133+ cells and analyzed for the CD44hi and CD2410 CSC population. Results showed that in the presence of TITE proportion of CSC was reduced to 0.7% compared to 4.9% in control culture without TITE (FIG. 111). Cultures containing B-CM also had reduced proportions of CD44hi/CD2410 CSC compared to control condition (2.1 vs. 4.9%).

### Example 8

### TITE Exhibits Cytotoxicity Against Multiple Cancer Cell Lines

Further, TITE were examined to determine if they show similar effect as seen in 3D cultures for various tumor cell lines. First, the effect of various doses (5%, 10% and 25%) B-CM or TITE on tumor cells including SKBR3, MB231, MiaPaCa-2, L3.6p1, CoLo-356, A431 and H292 was determined (FIG. 1I). At 5%, TITE mediated tumor lysis was very low to none, the cytotoxicity at 10% TITE ranged from 10-50% against various cell lines. The TITE mediated cytotoxicity was consistently high across multiple cell lines at 72 h by MTT assay. It was confirmed that the TITE mediated cytotoxicity in a large number of cell lines from various tumor types. A 25% dose of TITE showed highly significant cytotoxicity (p<0.005-p<0.0005) against MB231, MCF-7, SKBR3, MiaPaCa-2, L3.6p1, CoLo-356, HCT8, H292, A549, HN6 compared to B-CM at 72 hour. One of the head and neck cell lines H460 showed high cytotoxicity by both BCM and TITE while HN12 showed no killing by either B-CM or TITE (FIG. 1I).

### Example 9

### BATs Exhibit Enhanced Cytotoxicity at Tumor Cells Primed with TITE

Since 25% TITE was cytotoxic to various tumor targets, next priming of tumor cells with TITE (24 h exposure with 5% TITE) was studied to determine if the priming can enhance BATs mediated cytotoxicity. Continuous monitoring by Real Time Cell Analysis (RTCA) was used. TITE prepared from the coculture of MB231 cells+BATs from three normal donors (TITE1, TITE2 TITE3) was tested against the same cell line (MB231) or pancreatic cancer cell line MiaPaCa-2. Cytotoxicity was 2 fold higher with 5% TITE compared to BATs (2:1 E/T ratio) from all three donors for both cell lines. Similarly, cytotoxicity by BATs (2:1 E/T ratio) increased against MiaPaCa-2 or MB231 cells after being primed with 5% TITE overnight compared to BATs on unprimed MiaPaCa-2 or MB231 (FIG. 1i, top two graphs).

### Example 10

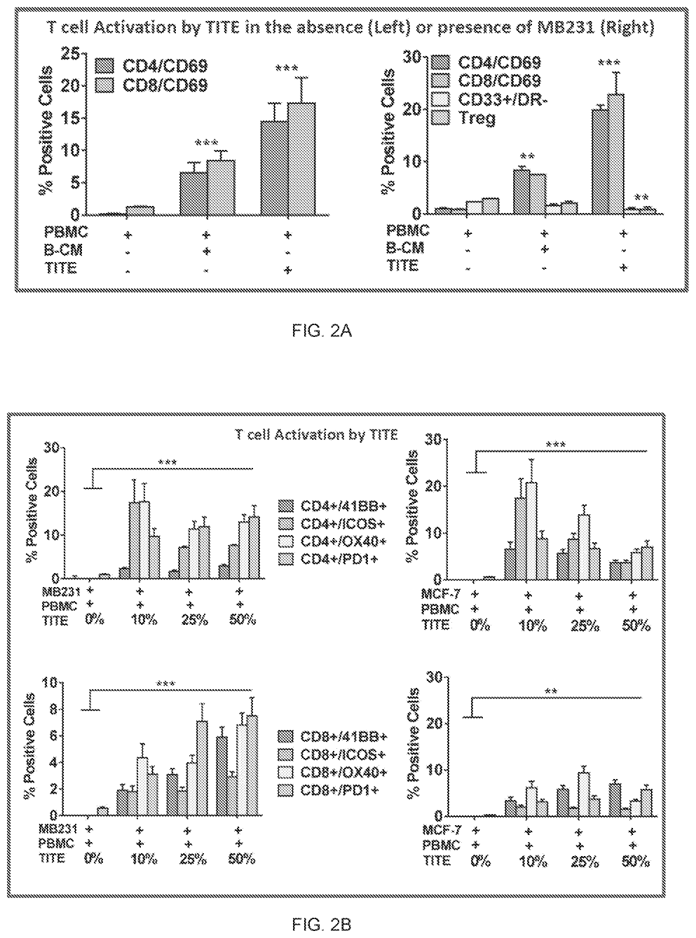

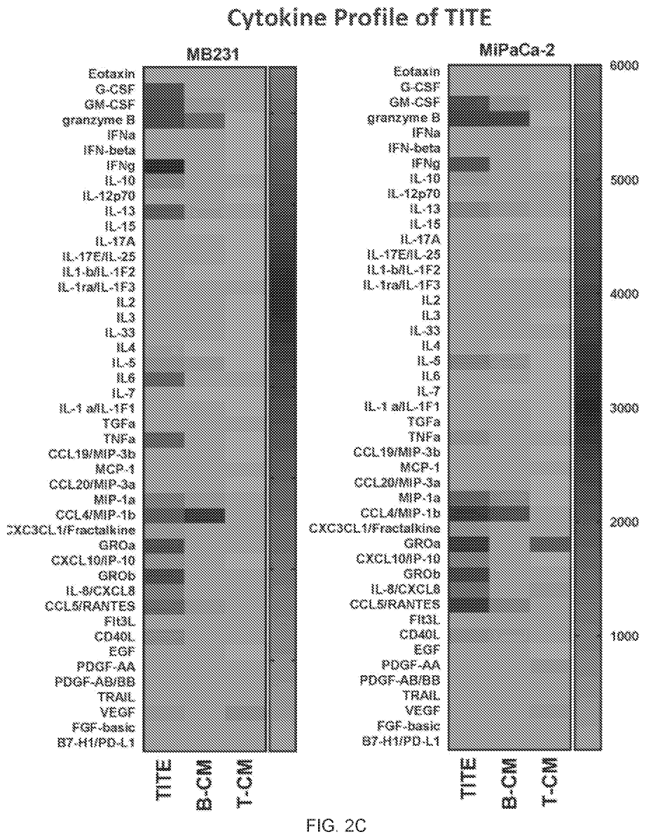

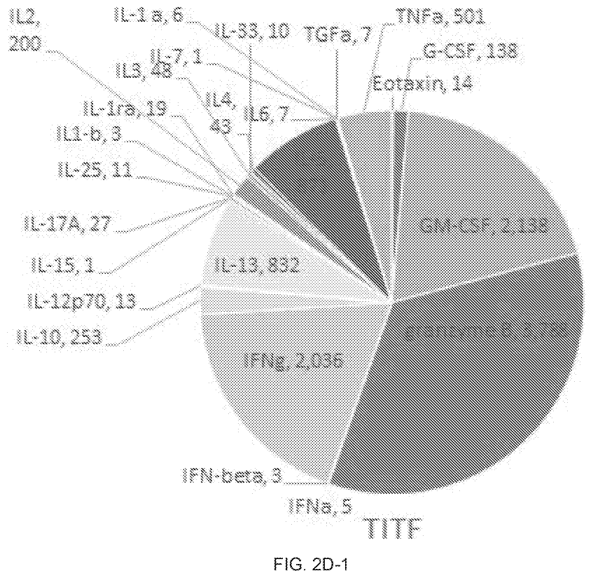

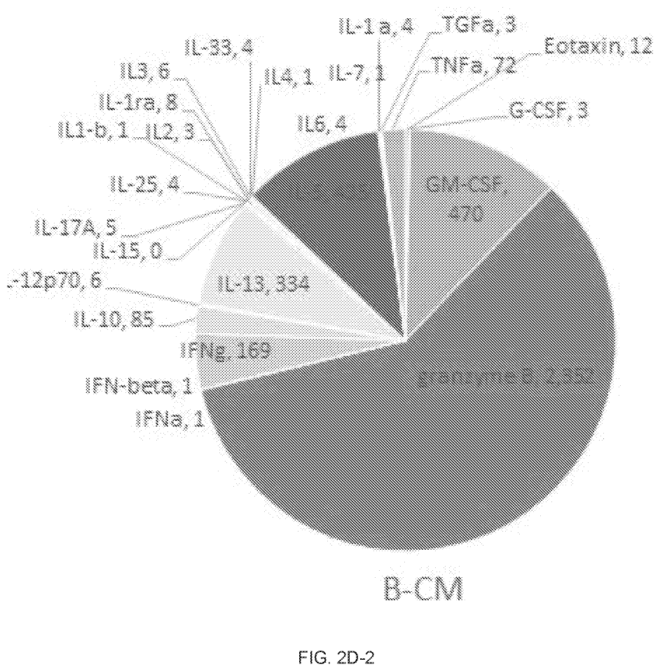

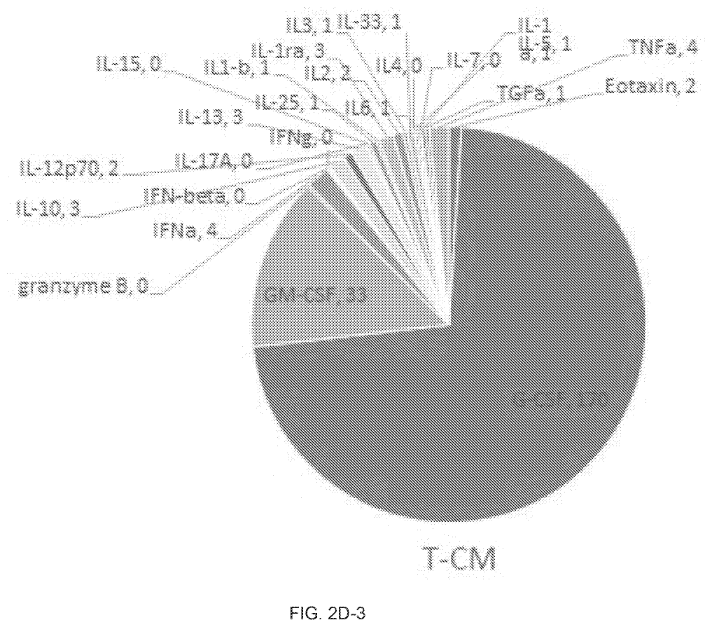

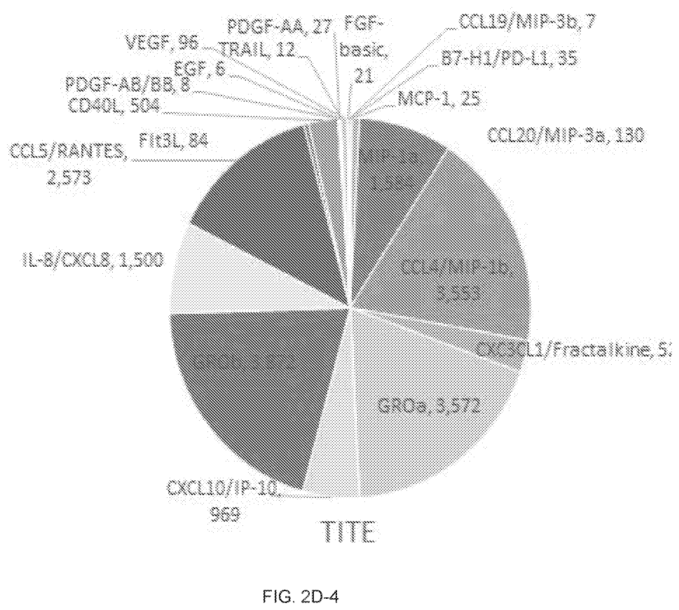

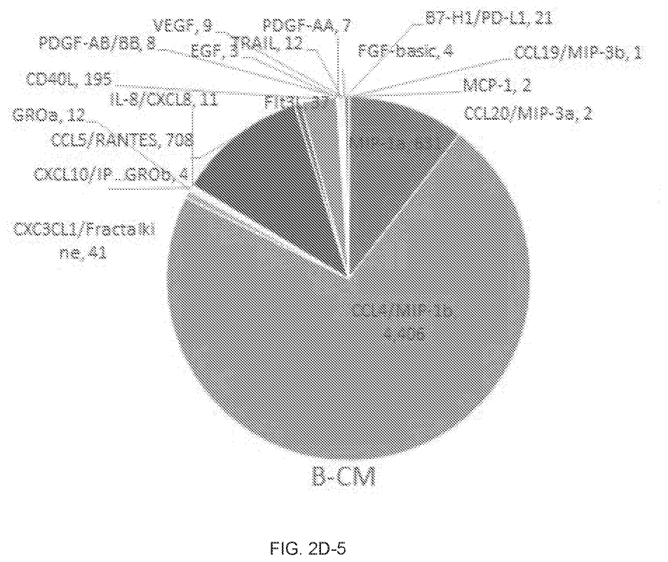

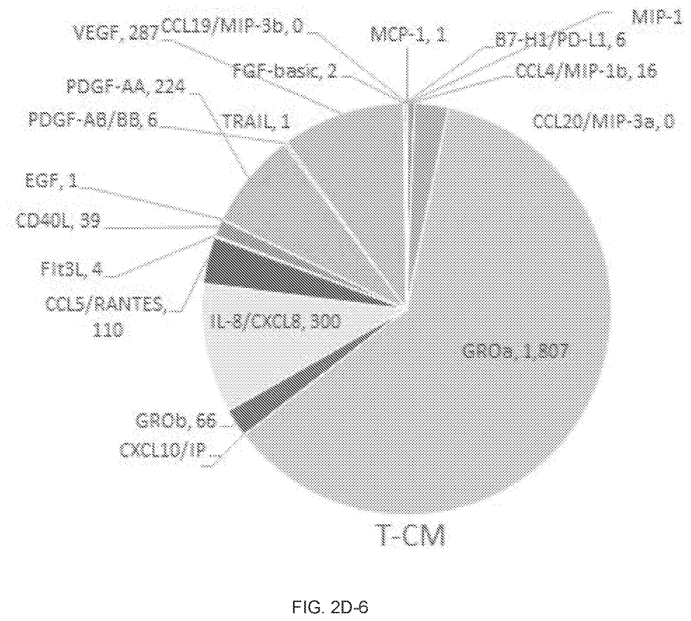

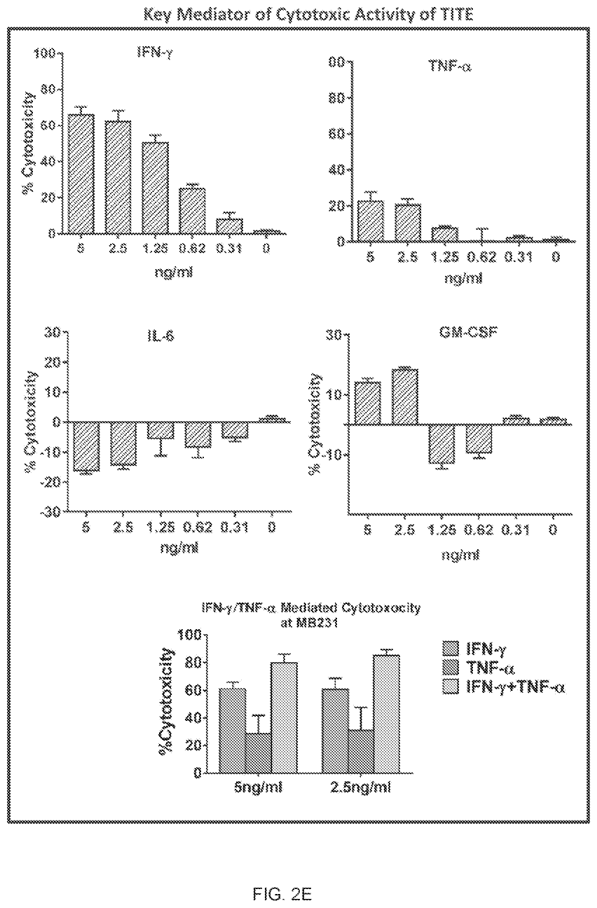

TITE Induces Activation and Proliferation of T cell Subsets in 3D Co-culture Phenotypic changes that occurred in the T cells in the presence of TITE at different time points was investigated. Expression of early activation marker CD69, immune-modulatory molecules 4-1BB, ICOS and checkpoint molecules PD1 and CTLA-4 were evaluated. Early activation marker, CD69 was upregulated in T cells isolated from the co-culture with tumor cells. In the presence of TITE the expression of CD69 was significantly higher on both CD4+ and CD8+ T cell sub-populations (FIG. 2A, left panel).

A significant increase in the expression of the activating co-stimulatory molecule 4-1BB, ICOS and PD-1 up to 5-fold on CD4+ T cells and 2-fold on CD8+ T cells in the presence of MB231 cells (p<0.0005) or MCF-7 cells (p<0.005-p<0.0005) was observed (FIG. 2B). Taken together, the data show that TITE induced activation of CD4+ and CD8+ T cells is reflected by increased expression of activation markers on T cells.

### Example 11

### TITE Inhibits MDSC and Tregs in 3D Co-Culture

Next, the effect of TITE on myeloid derived suppressor cells (MDSC) and Tregs in the TME was examined and compared to the T-CM or B-CM. The phenotype of monocyte co-cultured with tumor spheroids was characterized by analyzing the expression of cell surface markers by flow cytometry. There was a significant reduction in CD33+/CD11b+/HLA-DRMDSC (p<0.002) and CD4+/CD25+/CD127− Treg populations (p<0.001) in the presence of TITE compared to T-CM (FIG. 2A, Right panel).

### Example 12

### The Dominant Cytokines/Chemokines in TITE Show Th1 Profile

Soluble factors including cytokines, chemokines and growth factors in supernatants from tumor alone (T-CM), BATs alone (BCM) or tumor cells+ BATs co-culture (TITE) were measured using the Luminex multiplex technology.

It was observed that TITE differed in their cytokine/growth factor profile depending on the tumor cell line. However, tumor cell line MB231 co-cultured with BATs secreted high levels Th1 cytokines IFN-γ, TNF-α, Granzyme B, GM-CSF, G-CSF, proliferation inducing cytokines Flt3L, IL-2, IL-3, moderate levels of Th2 cytokines IL-10, IL-5, IL-6 and IL-13, and growth factors CD40L, VEGF and PDGF-AA. The levels of chemokines IP-10, MIP-1a, MIP-1b, RANTES, GRO-a, GRO-b, IL-8 also increased significantly in TITE compared to T-CM and B-CM levels of cytokine and chemokines (FIG. 2C). FIGS. 2D-1 through FIG. 2D-6 provide the values of cytokines (FIGS. 2D-1 through 2D-3), chemokines and growth factors (FIGS. 2D-4 through 2D-6) displayed in pie charts (for MiaPaCa-2 targets).

### Example 13

### IFN-γ, TNF-α and Granzyme B Mediated Tumor Cell Killing

The TITE generated from Tumor/BATs co-culture showed significantly higher levels of IFN-γ, TNF-α and GZB compared to B-CM and T-CM (FIG. 2E). Dose titration of showed cytotoxic activity at 2.5 ng/ml concentration of IFN-γ (˜60%) and TNF-α (˜20%) and combination of IFN-γ and TNF-α showed additive cytotoxic effect (˜80%). Since, GZB has already been shown to have potent lytic activity and play a major role on T cell mediated cytotoxicity, the cytotoxic activity of GZB was not tested. GZB is an active member in the granzyme family, which activates initiator caspases (such as caspases-8, -9, -10) and then promotes apoptosis through directly processing effector caspases-3 and -7 to promote apoptosis [10, 11].

### Example 14

### Phospho-Specific Protein Array Pattern Indicate JAK/STAT1 Signaling in TITE

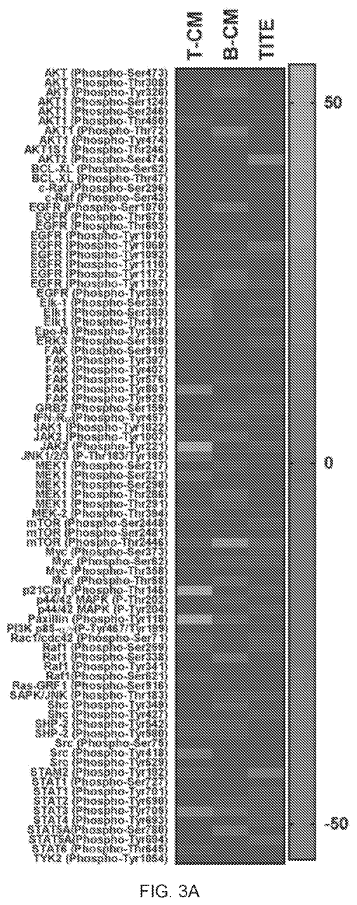

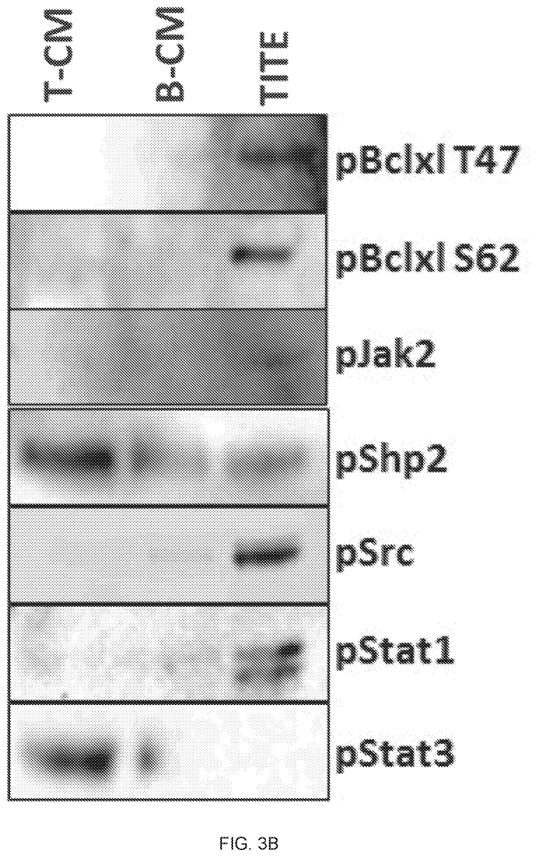

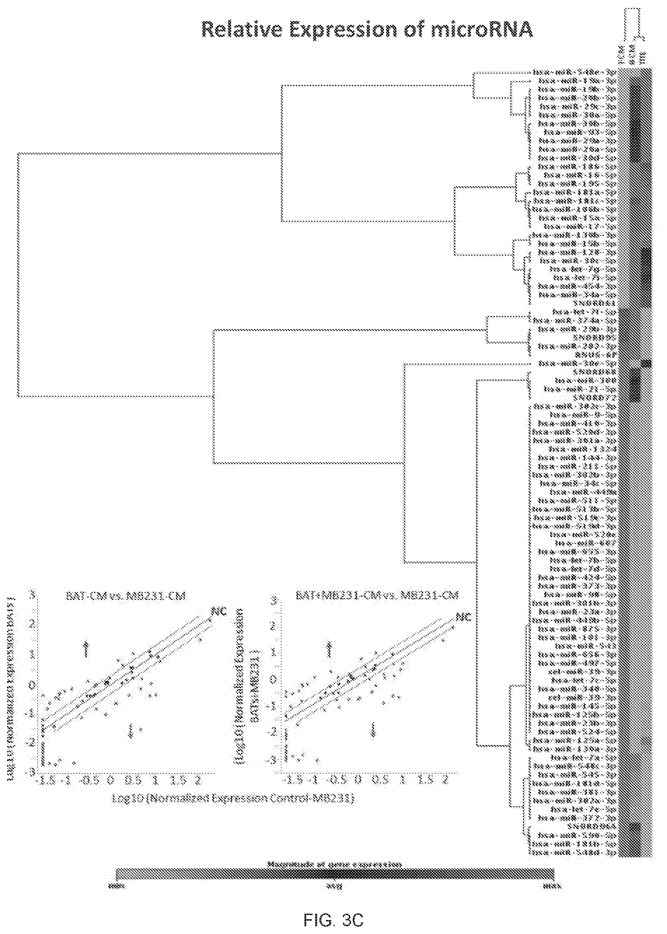

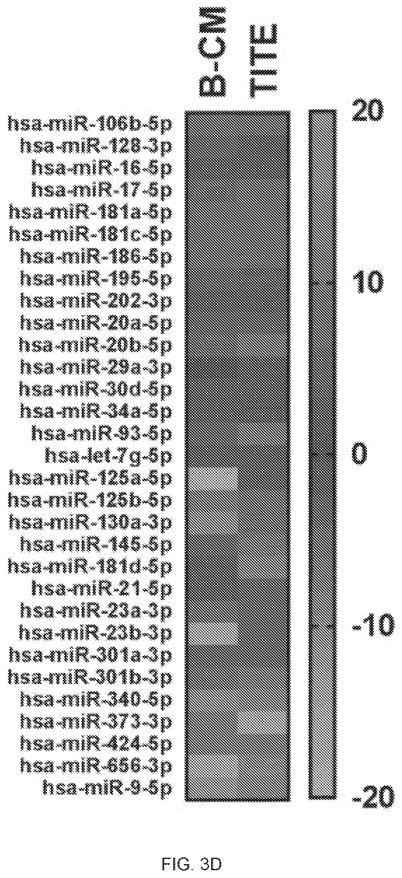

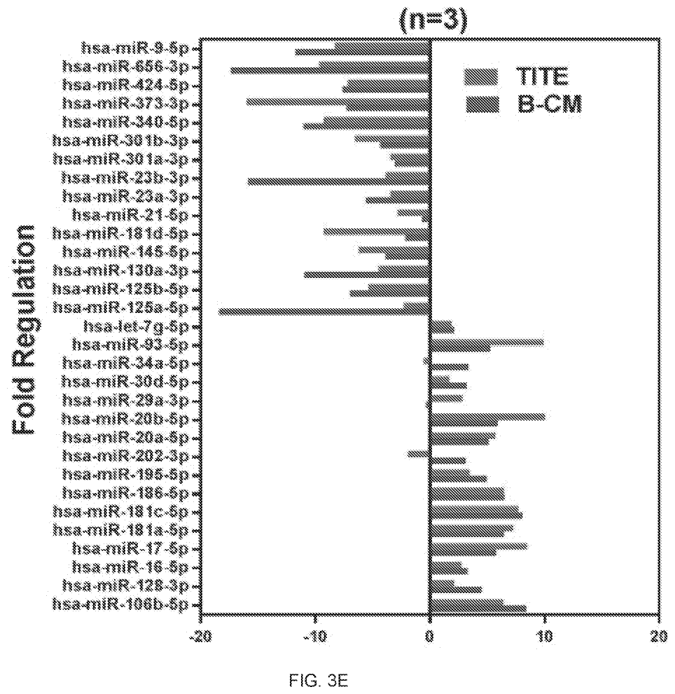

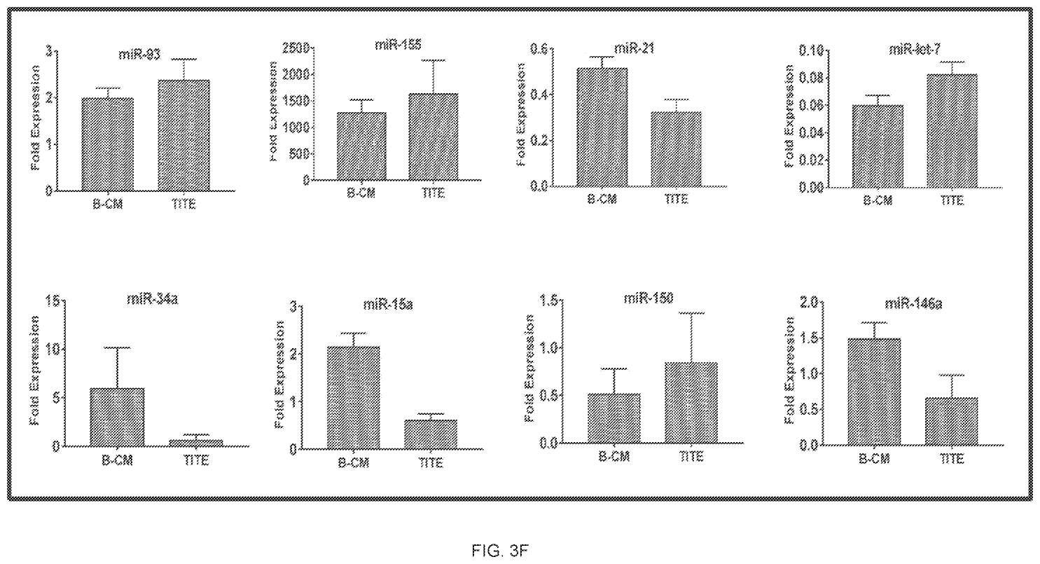

Phosphoantibody array data revealed increased expression of STAT1/STAT3/STAT5 in TITE and increased STAT3/STATE in B-CM and T-CM, STAT3 is common in all three TITEs, but STAT5 is specific to B-CM and TITE and STATE to T-CM (FIG. 3A). In T-CM, there were increased expression of phospho-STAT3 (Tyr705) and phospho-STAT6 but low expression of STAT1 compared to B-CM or TITE. Since, T-CM shows high levels of VEGF and PDGF in may drive tumor cell growth, survival and tumor promoting Th2 cytokines through JAK/STAT3 or JAK/STAT6 signaling pathway. Selected signaling molecules were analyzed by the western blot (FIG. 3B) corroborate the phospho-signaling array data.

### Example 15

### TITE Show Immune Activating miRNA Expression Profile

To understand the role of miRNAs in the alteration of cellular and tumor microenvironment plasticity maintenance of breast cancer, exosomal RNAs from culture supernatants of various culture conditions using MB-231 cell line and BATs (T-CM, B-CM and TITE) were used for the miRNA profiling. The panel consisted of 84 miRNA, there were 72 miRNAs that were differentially expressed (fold-change >2 or <−2, P<0.05) in TITE (n=3) or B-CM compared to T-CM (n=3), there were 12 common miRNAs in all three groups. At the fold change cut-off of 1.5, the miRNA array revealed 16/84 miRNA significantly up-regulated and 56 down-regulated miRNAs (FIG. 3C). The heat maps indicated the number of miRNAs that were differentially regulated in different conditioned media (see FIGS. 3D and 3E). Many miRNAs that were down regulated in TCM samples were mostly associated with pro-apoptotic pathways. Among the 72 miRNAs common to B-CM and TITE, approximately 19% (n=16) were upregulated and ˜66% (n=56) were downregulated in both groups (FIGS. 3C-3E). Selected miRNA (miR-21, miR-15, miR-34, miR-93 and miR-let-7) were validated by qRT-PCR. Representative qRT-PCR data of miR-93 and miR-let-7 show concordance with miRNA array data (FIG. 3F). Since two important immune related miRs, miR-155, miR-150 and miR-146, were not present in a 84-panel miR array, qRT-PCR for miR-155 and miR-146 showed upregulation of miR-155 and downregulation of miR-146 (FIG. 3F).

### Example 16

### TITE Treated Mice Show Significantly Reduced Tumor Growth

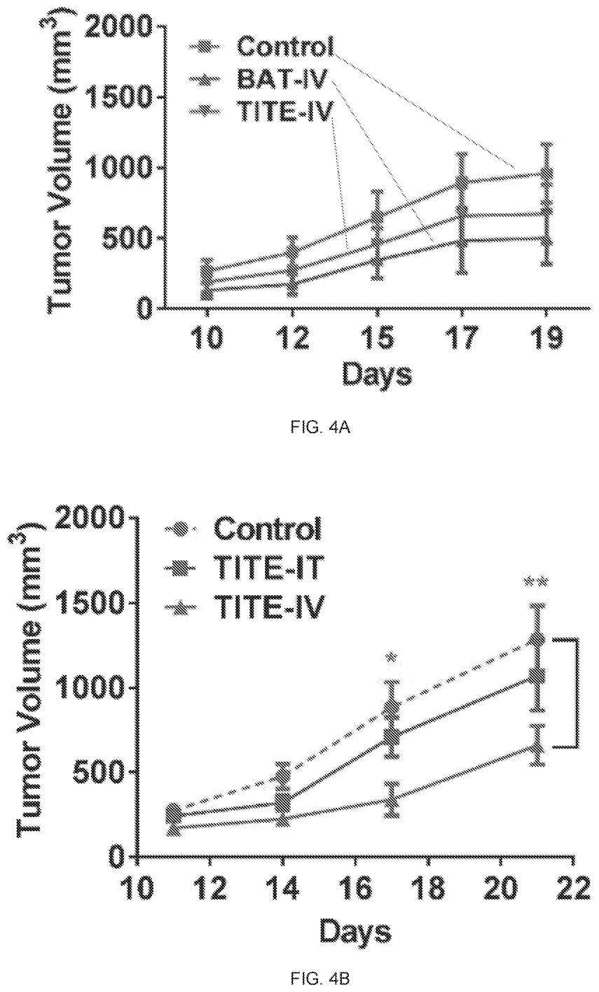

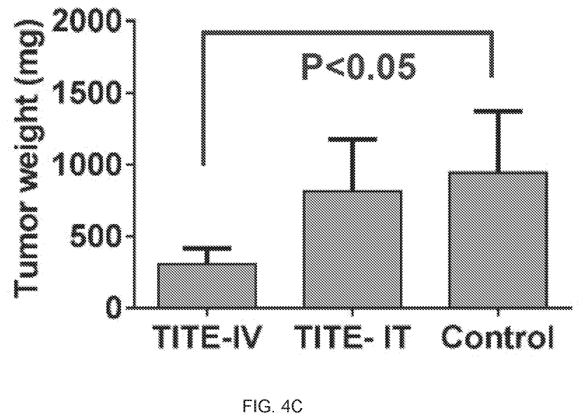

The MB-231 tumor cell line was injected into the flanks of ICR-SCID mice (n=5/group). After tumors become palpable, tumor-bearing mice were split into three groups, group 1 was treated with IV injections of BATs, and group 2 was treated with IV injections of TITE and group 3 with IV injections of vehicle 2×/week for 3 weeks. Tumor growth was delayed after 3 weeks in both BATs and TITE treated mice (FIGS. 4A-4C) compared to vehicle injected control mice. This delay was compared to a 3× injection/week schedule. Also, the route of injection (IV versus intra-tumorally (IT) treated mice) was compared to see if there was comparable tumor growth delay (n=10). Mice were treated with TITE (IV vs. IT) after tumor become palpable 3×/week for 2-3 weeks. Tumor growth was significantly delayed (tumor size, p<0.05; tumor volume p<0.003) after 2 weeks when TITE was injected IV (FIGS. 4A-4C) compared to IT treated or control mice, however, no tumor regression or cures were noted. No sign of toxicity was observed in heart, lung, liver and spleen in TITE treated mice examined by H&E staining of tissue sections.

Referring to FIGS. 4A-4C, the MB-231 breast cancer cell line was injected into the flanks of ICR-SCID mice (n=5/group), tumor-bearing mice were treated with IV injections of BATs, TITE and vehicle 2×/week for 3 weeks (FIG. 4A). In FIG. 4B, tumor-bearing mice treated with IV vs IT injections TITE and vehicle 3×/week for 3 weeks show significant delay in tumor volume (p<0.003) as well as tumor size (p<0.05) in FIG. 4C.

### Example 17

### Staining of TITE Treated Tumors Show Increased Duct Formation at the Margins

Control, TITE-IT and TITE-IV treated tumor sections from Example 16 were stained for myeloperoxidase (for granulocytes), F4/80 (for macrophages) and H&E. The representative H&E staining in xenograft tumors (scale bar=2 mm) show that IV treated tumors show duct formation at the margins and central necrotic region, the IT treated tumors appear large in size but show large necrotic region in the center, compared to solid center in the control mice. Staining for macrophages appear to be at the margin for all control and treated tumors, but granulocytes in IT treated tumors surround the tumor cell islands and IV treated tumors show focal infiltration compared to the control tumors with marginal and diffused infiltration of granulocytes.

### Discussion of Examples

The tumor/BATs-derived TITE initiates the paracrine signaling responsible for immune activation and anti-tumor activity through growth factors, cytokines, and chemokines. The TITE, enriched in Th1 cytokine/chemokines, showed significant destruction of tumor spheres at the concentration of just 10% (TITE prepared from of BATs and tumor cells co-culture at 25:1 ratio) with complete destruction at 50% concentration.

Similar killing of all cell populations was observed by TITE when tumor cells were incubated with MDSC and/or Tregs. Moreover, TITE prepared from a single cell line was cytotoxic against multiple cell lines from multiple tumor types indicating that TITE can serve as a universal anti-cancer drug. In contrast to its cytotoxic effects on tumor and immune suppressive cells, TITE induced nodal proliferation of LECs, and activation and proliferation of T cells suggesting cell specific effects of TITE. There was a significant reduction (p<0.002) in MDSC and Treg populations in the presence of TITE, and a significant increase in activation (CD69, p<0.0005) and co-stimulatory molecules (4-1BB/ICOS/OX40, p<0.005-0.0005) CD4+ and CD8+ T cells in the presence of both MB231 and MCF-7 cells. Without being bound to any one theory, these findings suggest that TITE mediates high level cytotoxic activity against a variety of tumor cell lines, inhibits of immune suppressor cells (Tregs and MDSC) in the tumor microenvironment, and promotes activation and proliferation of T cells and LECs.

To understand the mechanism triggering release of anti-tumor and immune activating mediators of TITE, multiplex cytokine/chemokine array, phospho-signaling array, and miRNA array analyses were performed. The cytokine profile of TITE from MB231 and BATs co-culture shows enrichment of Th1 cytokines (IFN-γ, TNF-α, Granzyme B), T cell and monocytes recruiting chemokines (IP-10, MIP-1a, MIP-1b, RANTES), monocytes activating cytokines (GM-CSF, TNF-α, CD40L), and Th2 cytokines (IL-10, IL-5, and IL-13) compared to the levels of cytokine and chemokines in B-CM and T-CM. Cytokine profiling data suggest that TITE is enriched in dual functioning key cytokines (immune modulating cytokines [IFN-γ, TNF-α, GM-CSF] and tumor killing cytokines [IFN-γ, TNF-α, Granzyme B]). The T-CM showed increased levels of chemokine fractalkine (CX3CL1) compared to B-CM and TITE that has been shown to support the cell survival of tumor cells by down regulating pro-apoptotic BID and BAX [12]. Since TITE comprises the released factors as a function of tumor stimulated BATs either through activation induced release or the release of factors due to activating induced cell death, the phospho-antibody array data revealed that the prominent STAT signaling in activated BATs is mediated by STAT1 and STAT5. Both, STAT1 and STAT5 are activated by a number of different ligands, including IFN-γ, EGF, PDGF and IL-6 by phosphorylation at both Tyr701 and Ser727 [13-15]. The phosphorylation of STAT1 at Tyr701/Ser727 induces STAT1 dimerization, nuclear translocation and DNA binding for full transcriptional activity and biological function [16]. The release of Th1 cytokines appears to be through JAK/STAT1 signaling in BATs while Th2 signaling is likely to be via downstream of CD3 (via engagement with bispecific antibody) through PI3K/AKT/mTOR/STAT3 signaling [17]. TITE showed low levels of, negative regulator of JAKSTAT1, SHP-2 [18] compared to T-CM indicating reduced inhibition of JAK/STAT1 signaling. The prominent STAT signaling in T-CM appears to be mediated by phospho-STAT3 (Tyr 705)/phospho-STAT6 and a low expression of phospho-STAT1 in T-CM compared to B-CM or TITE. The STAT3 and STATE are signaling pathways in tumors induced by upstream regulators such as VEGF, EGF, PDGF, and IL-6 [19-23]. Without being bound to any one theory, the presence of high levels of VEGF and PDGF in T-CM can promote JAK/STAT3 or JAK/STAT6 signaling to drive tumor cell growth and survival [19, 23]. VEGF has been shown to promote breast and lung cancer stem cell (CSC) self-renewal via VEGF receptor-2 (VEGFR-2)/STAT3-mediated upregulation of Myc [24-26]. VEGF exposure prior to tumor cell injection has been shown to increase breast cancer metastasis from orthotopic primaries [26], and VEGFR-2 inhibition mitigates VEGF-mediated STAT3 activation and sphere formation [21, 22, 25, 26]. These findings corroborate with cytokine/chemokine/growth factor array data as upstream regulators of downstream regulation of transcription factors and positive feedback loop of cytokine/chemokine induction and release.

The miRNA array data show that highly upregulated miRNAs in TITE and B-CM (e.g., miR-16-5p, miR-17-5p, miR-195-5p, miR-20a-5p, miR-93-5p, miR-155, miR-181a-5p, miR-181c-5p, miR186-5p, miR-106a-5p) are associated with T cell function and activation and activation-induced cell death [17, 27-29]. Since some of the immune function related miRNAs were not present in the 87-miRNA panel, qRT-PCR for miR-155, miR-150, miR-146a, and miR-34a was performed. The data show that miR-150 and miR-146a were downregulated in both B-CM and TITE, while miR-155 was highly upregulated in TITE. The miR-155 and miR-146a act as a positive and negative regulators of immune response through the direct targeting of IRAK1 and TRAF6, and SOCS1 and BCL6 [30], respectively. Previous reports have shown miR-155, miR-125a/b are associated with the activation of macrophages via TLR signaling to sustain M1-like TAM activation, whereas miR-146 repress M1 TAM [30]. The miR-155 expression has also been shown to regulate differentiation of T helper cells in favor of Th1 phenotype, the maturation and activation of CTLs into effectors, and miR-17/92, miR-30b and miR-155 promote memory cell subsets [31]. In contrast, miR-130/301 and miR-146a have displayed inhibiting effects on CTL immune responses [32]. Similar to immune response regulation, miRNAs play roles in the regulation of tumor growth, invasion and metastasis, miR-25-93-106b cluster has been shown to regulate both CXCL12 and PD-L1 in pancreatic cancer patients resistant to PD-1 inhibition [33]. The miR-17 (upregulated in TITE) has been shown to inhibit invasion and metastasis of MB231 cells using conditioned medium from miR-17/20-overexpressing MCF-7 cell line [34]. The miRs down regulated in TITE (miR-125a/b miR-146a miR-150a miR-21, miR-301a and miR-301b) have been shown to promote tumor growth and migration by targeting TGFBR2 to modulate TGF-β signaling pathway in colorectal carcinoma [35].

Cell based therapy, particularly CAR T cell therapy, offers a novel and potent therapeutic modality, but has shown limited activity in solid tumors. These limitations are attributed to their poor penetration in solid mass, loss of activity in immune suppressive TME and “off tumor on target” toxicity such as cytokine release syndrome (CRS) and life-threatening cytokine storms [36-38]. The presently disclosed TITE serve as potent anti-tumor and immune activating drugs that can provide more control in key processes and overcoming the therapeutic limitations. Thus, the therapeutic advantages of TITE are manifold to combat cancers, such as metastatic breast cancer, and include but are not limited to: 1) eliminating the preparation and waiting time of patient's T cells collection for activation and expansion before the treatment initiation, 2) providing a more potent and effective treatment than patient's own T cells, since TITE can be generated from normal, healthy donor T cells, 3) providing a ready off-the-shelf product, and 4) providing a treatment that is highly cost effective compared to cell therapy. It is expected that the presently disclosed TITE based approach can not only be more effective by targeting “tumor” and altering the “TME” but can also overcome the challenges of tumor heterogeneity and mutational burden. The development of a broad tumor-specific adaptive immune response due to epitope-spreading as a consequence of tumor destruction and inflammation has long been proposed to be a secondary mechanism underlying the potency of immunotherapy. This strategy can maximize anti-tumor efficacy and promote long-term immunity in cancers, such as metastatic breast cancer, leading to superior outcomes for patients fighting this disease and durable anti-tumor responses. The presently disclosed innovative cell-free therapeutic platform focuses on long lasting anti-tumor immunity through induction of in situ immunization.

### REFERENCES

All references listed below, as well as all references cited in the instant disclosure, including but not limited to all patents, patent applications and publications thereof, scientific journal articles, and database entries (e.g., GENBANK® and UniProt biosequence database entries and all annotations available therein) are incorporated herein by reference in their entireties to the extent that they supplement, explain, provide a background for, or teach methodology, techniques, and/or compositions employed herein.
1. Lum, L. G., et al., Clin Cancer Res, 2015. 21(10): p. 2305-14.
2. Vaishampayan, U., et al., Prostate Cancer, 2015. 2015: p. 285193.
3. Thakur, A., et al., Oncoimmunology, 2018. 7(12): p. e1500672.
4. Thakur, A. and L. G. Lum, Oncoimmunology, 2016. 5(3): p. e1055061.
5. Sen, M., et al., J Hematother Stem Cell Res, 2001. 10(2): p. 247-60.
6. Thakur, A., O. Norkina, and L. G. Lum, Cancer Immunol Immunother, 2011. 60(12): p. 1707-20.
7. Thakur, A., et al., Cancer Immunol Immunother, 2012. 61(4): p. 497-509.
8. Hernandez, C., P. Huebener, and R. F. Schwabe, Oncogene, 2016.35(46): p. 5931-5941.
9. Thakur, A., et al., J Transl Med, 2013. 11: p. 35.
10. Pardo, J., et al., J Cell Biol, 2004. 167(3): p. 457-68.
11. Goping, I. S., et al., Immunity, 2003. 18(3): p. 355-65.
12. White, G. E. and D. R. Greaves, Arterioscler Thromb Vasc Biol, 2012. 32(3): p. 589-94.
13. Zhang, Y. and Z. Liu, Discov Med, 2017. 24(130): p. 19-29.
14. Rauch, I., M. Muller, and T. Decker, JAKSTAT, 2013. 2(1): p. e23820.
15. Able, A. A., J. A. Burrell, and J. M. Stephens, Biology (Basel), 2017. 6(1).
16. Dimberg, A., et al., Blood, 2003. 102(1): p. 254-61.
17. Hoefig, K. P. and V. Heissmeyer, J Cell Biol, 2018. 217(8): p. 2615-2631.
18. Liu, W., et al., Oncotarget, 2017. 8(5): p. 7586-7597.
19. Su, Y. L., et al., Int J Mol Sci, 2018. 19(6).
20. Zhao, D., et al., Oncogene, 2014. 34: p. 3107.
21. Bogin, L. and H. Degani, Cancer Res, 2002. 62(7): p. 1948-51.
22. Lee, T. H., et al., J Biol Chem, 2003. 278(7): p. 5277-84.
23. Binnemars-Postma, K., et al., FASEB J, 2018. 32(2): p. 969-978.
24. Lee, H., et al., Cancer Cell, 2009. 15(4): p. 283-93.
25. Zhao, G., et al., Oncol Rep, 2016. 35(3): p. 1787-95.
26. Zhao, D., et al., Oncogene, 2015. 34(24): p. 3107-19.
27. Torri, A., et al., J Biol Chem, 2017. 292(7): p. 2903-2915.
28. Podshivalova, K. and D. R. Salomon, Crit Rev Immunol, 2013. 33(5): p. 435-76.
29. Rodriguez-Galan, A., L. Fernandez-Messina, and F. Sanchez-Madrid, Front Immunol, 2018. 9: p. 2148.
30. Testa, U., et al., Noncoding RNA, 2017. 3(3).
31. Liang, Y., H. F. Pan, and D. Q. Ye, J Leukoc Biol, 2015. 97(3): p. 487-97.
32. Paladini, L., et al., J Exp Clin Cancer Res, 2016. 35: p. 103.
33. Cioffi, M., et al., Oncotarget, 2017. 8(13): p. 21609-21625.
34. Yu, Z., et al., Proc Natl Acad Sci USA, 2010. 107(18): p. 8231-6.
35. Soleimani, A., et al., J Cell Physiol, 2019.
36. Porter, D. L., et al., N Engl J Med, 2011. 365(8): p. 725-33.
37. Guedan, S., M. Ruella, and C. H. June, Annu Rev Immunol, 2018.
38. Watanabe, K., et al., Front Immunol, 2018. 9: p. 2486.
39. Lum, L. G., et al., Biology of Blood Marrow Transplantation 19: 925-933, 2013.
40. Lum, L. G., et al., Biol of Blood and Marrow Transplantation 22: 869-878, 2016.

While the presently disclosed subject matter has been disclosed with reference to specific embodiments, it is apparent that other embodiments and variations of the presently disclosed subject matter may be devised by others skilled in the art without departing from the true spirit and scope of the presently disclosed subject matter.

1. A method for treating cancer in a subject in need thereof, the method comprising administering to the subject an effective amount of a composition comprising Tumor-Targeting Effectors (TITE) derived from a culture comprising a bispecific antibody (BiAb) armed activated T cell (BAT) and a cancer cell, to thereby treat cancer in the subject.

2. (canceled)

3. The method of claim 1, wherein the cancer cell is from a cancer selected from the group consisting of a breast cancer, a pancreatic cancer, a prostate cancer, a brain cancer, a lung cancer, an ovarian cancer, a liver cancer, a leukemia, non-Hodgkin's lymphoma and multiple myeloma.

4-5. (canceled)

6. The method of claim 1, wherein the BiAb used to arm the activated T cell is a chemically heteroconjugated bispecific antibody or a recombinant bispecific antibody of any configuration.

7. The method of claim 1, wherein the activated T cells are produced from an apheresis product.

8. The method of claim 7, wherein the activated T cells are produced from an apheresis product by anti-CD3 stimulation in the presence of IL-2, optionally at a range of about 20 to about 200 IU/ml, or wherein co-stimulated T cells are produced from an apheresis product by co-stimulation with anti-CD³/anti-CD28 coated beads, optionally in the presence of IL-2 at a range of about 20 to about 200 IU/ml, optionally at bead to cell ratios from about 1:3 to about 3:1.

9. The method of claim 1, wherein the culture comprises peripheral blood mononuclear cells, unfractionated CD3+ T cells, CD4+ T cells, or CD8+ T cells.

10. The method of claim 1, wherein the TITE comprise secreted agents selected from the group consisting of a Th1 cytokine, a proliferation inducing cytokine, a Th2 cytokine, a chemokine, a growth factor, and any combination thereof.

11. The method of claim 10, wherein:
(a) the Th1 cytokine is selected from the group consisting of IFN-γ, TNF-α, Granzyme B, GM-CSF, G-CSF, and combinations thereof;
(b) the proliferation inducing cytokine is selected from the group consisting of Flt3L, IL-2, IL-3, and combinations thereof;
(c) the Th2 cytokine is selected from the group consisting of IL-10, IL-5, IL-6, IL-13, and combinations thereof; and/or
(d) the growth factor is selected from the group consisting of CD40L, VEGF, PDGF-AA, and combinations thereof.

12-13. (canceled)

14. The method of claim 1, wherein the composition comprising TITE comprises a miRNA.

15. The method of claim 14, wherein the miRNA is selected from the group consisting of miR-16-5p, miR-17-5p, miR-195-5p, miR-20a-5p, miR-93-5p, miR-155, miR-181a-5p, miR-181c-5p, miR186-5p, and miR-106a-5p.

16-18. (canceled)

19. A pharmaceutical composition comprising, consisting essentially of, or consisting of an effective amount of a composition comprising Tumor-Targeting Effectors (TITE) derived from a culture comprising a bispecific antibody armed activated T cell (BAT) and a cancer cell.

20. (canceled)

21. The composition of claim 19, wherein the cancer cell is from a cancer selected from the group consisting of a breast cancer, a pancreatic cancer, a prostate cancer, a brain cancer, a lung cancer, an ovarian cancer, a liver cancer, a leukemia, non-Hodgkin's lymphoma and multiple myeloma.

22-23. (canceled)

24. The composition of claim 19, wherein the BiAb used to arm the activated T cell is a chemically heteroconjugated bispecific antibody or a recombinant bispecific antibody of any configuration.

25. The composition of claim 19, wherein the activated T cells are produced from an apheresis product.

26. The composition of claim 25, wherein the activated T cells are produced from an apheresis product by anti-CD3 stimulation in the presence of IL-2, optionally at a range of about 20 to about 200 IU/ml, or wherein co-stimulated T cells are produced from an apheresis product by co-stimulation with anti-CD³/anti-CD28 coated beads, optionally in the presence of IL-2 at a range of about 20 to about 200 IU/ml, optionally at bead to cell ratios from about 1:3 to about 3:1.

27. The composition of claim 19, wherein the culture comprises peripheral blood mononuclear cells, unfractionated CD3+ T cells, CD4+ T cells, or CD8+ T cells.

28. The composition of claim 19, wherein the TITE comprise secreted agents selected from the group consisting of a Th1 cytokine, a proliferation inducing cytokine, a Th2 cytokine, a chemokine, a growth factor, and any combination thereof.

29. The composition of claim 19, wherein:
(a) the Th1 cytokine is selected from the group consisting of IFN-γ, TNF-α, Granzyme B, GM-CSF, G-CSF, and combinations thereof;
(b) the proliferation inducing cytokine is selected from the group consisting of Flt3L, IL-2, IL-3, and combinations thereof;
(c) the Th2 cytokine is selected from the group consisting of IL-10, IL-5, IL-6, IL-13, and combinations thereof; and/or
(d) the growth factor is selected from the group consisting of CD40L, VEGF, PDGF-AA, and combinations thereof.

30-31. (canceled)

32. The composition of claim 19, wherein the composition comprising TITE comprises a miRNA.

33. The composition of claim 32, wherein the miRNA is selected from the group consisting of miR-16-5p, miR-17-5p, miR-195-5p, miR-20a-5p, miR-93-5p, miR-155, miR-181a-5p, miR-181c-5p, miR186-5p, and miR-106a-5p.

34. (canceled)

35. A method of preparing a composition for treating cancer, the method comprising (a) culturing T cells and cancer cells with a bispecific antibody to provide a culture comprising a complex comprising cancer cells and activated T cells (ATC) wherein one binding domain of the bispecific antibody binds to an antigen on the T cells and a second binding domain of the bispecific antibody binds an antigen on the cancer cells; and (b) isolating media from the culture, wherein the media comprises BAT Induced Tumor-Targeting Effectors (TITE), to thereby provide a composition for treating cancer.

36-52. (canceled)


SEQUENCE LISTING (11 sequences)

SEQ ID NO: 1 - DNA, 22 bp, Homo sapiens
uagcagcacguaaauauuggcg

SEQ ID NO: 2 - DNA, 23 bp, Homo sapiens
caaagugcuuacagugcagguag

SEQ ID NO: 3 - DNA, 21 bp, Homo sapiens
uagcagcacagaaauauuggc

SEQ ID NO: 4 - DNA, 23 bp, Homo sapiens
uaaagugcuuauagugcagguag

SEQ ID NO: 5 - DNA, 23 bp, Homo sapiens
caaagugcuguucgugcagguag

SEQ ID NO: 6 - DNA, 24 bp, Homo sapiens
uuaaugcuaaucgugauagggguu

SEQ ID NO: 7 - DNA, 23 bp, Mus musculus
uuaaugcuaauugugauaggggu

SEQ ID NO: 8 - DNA, 23 bp, Homo sapiens
aacauucaacgcugucggugagu

SEQ ID NO: 9 - DNA, 22 bp, Homo sapiens
aacauucaaccugucggugagu

SEQ ID NO: 10 - DNA, 22 bp, Homo sapiens
caaagaauucuccuuuugggcu

SEQ ID NO: 11 - DNA, 23 bp, Homo sapiens
aaaagugcuuacagugcagguag